(19) United States
(12) Patent Application Publication
(10) Pub. No.: US 2019/0237168 A1
(43) Pub. Date: Aug. 1, 2019

(54) Abiotic Intelligence-Rendered Pay

(71) Applicant: Anuthep Benja-Athon, New York, NY (US)

(72) Inventor: Anuthep Benja-Athon, New York, NY (US)

(73) Assignee: Anuthep Benja-Athon, New York, NY (US)

(21) Appl. No.: 15/881,805

(22) Filed: Jan. 29, 2018

(51) Int. Cl. G16H 10/60; G16H 50/70; G06Q 20/38

(52) U.S. Cl. CPC G16H 10/60; G16H 30/20; G06Q 20/3821; G16H 50/70

(57) ABSTRACT

The system of networked repositories for precisely and accurately focusing physical movements and locations of global commodities and services and related financial transactions by rendering compilations of relations of commodities and services and related tallied values and costs and providing digital tokens for rendering and security for protecting the compilations and transactions.

This is a continuation-in-part of Ser. No. 15/495,164 entitled Abiotic Intelligent-Rendered Oracle filed Apr. 24, 2017, Ser. No. 14/992,773 entitled Creation of Abiotic-Biotic Civilization filed Jan. 11, 2016, Ser. No. 14/606,956 entitled Billion Recounts of Events Ahead of Days filed Jan. 27, 2015, Ser. No. 15/292,080 entitled EM Oracle filed Oct. 12, 2016, Ser. No. 12/592,714 entitled the Healthcare Exchange IV filed Dec. 2, 2009, Ser. No. 12/322,529 entitled the Healthcare Exchange II filed Feb. 4, 2009, Ser. No. 12/080,433 entitled The Healthcare Exchange filed Apr. 3, 2008 and Ser. No. 11/344,485 entitled Physicians-patients-health-care-buyers accounting system filed Feb. 1, 2006.

### FIELD OF THE INVENTION

The devices-gene hybrid for controlling transactions, movements and locations of global commodities and services.

### BACKGROUND OF THE INVENTION

The present civilization is just in the dawn of the Internet-rendered evolution. The status quo accesses, distributions, procurements and consumptions of all industrial goods and services by all people and businesses are random, haphazard, archaic and not cost-effective. Consequently, $trillions per annum are wasted and lost by said people and businesses causing low standard of living and quality of life, unavoidable earlier morbidity and mortality. The present invention rendering processor units-genes hybrid-rendered logistics for precisely and accurately focusing physical movements and locations of useful and indicated commodities and services and related financial transactions to people thereby improving and optimizing their standard and quality of living and increasing their health and longevity.

### SUMMARY OF THE INVENTION

The system of individual groups of networked repositories for precisely and accurately focusing physical movements and locations of global commodities and services and related financial transactions by said individual groups rendering compilations of relations of use and optimal continuums of commodities and services relating to compilations of relations of digital diagnosis-specific snippets of relational chronologically ordered health and healthcare data and related relational profile data and related tallied values and costs. The system provides digital tokens for rendering and security for protecting said compilations and transactions.

### DESCRIPTION OF THE PREFERRED EMBODIMENTS

The preferred embodiments of the present invention are thoroughly described in details in the Claims of the Invention herein.

The genes cited herein are human, animal, virus, bacterial, fungal and parasite and other living genes.

Although several interactive network visual-hierarchied data visualizations of visual-hierarchied flowingData in earlier patent applications have been used but not described herein, it will be appreciated by and obvious to those skilled in the art and science that a myriad of interactive network visualizations of visual-hierarchied flowingData such as, but not limited to, various interactive spectrums and gradients of connected visual-hierarchied data points, interactive graphs and interactive network visualizations of flowingdata of connected visual-hierarchied data points such as, but not limited to, tree traversals and adaptations, variations and derivatives will be made and used to achieve and enhance the objectives of the present invention without departing from the spirit and scope of the specification of the present invention.

Although various preferred embodiments of the present invention have been described, it will be appreciated by and obvious to those skilled in the art and science that adaptations, variations and derivatives of said embodiments are and will be made and achieved for animals without departing from the spirit and scope of the present invention.

1. The system of networked communication devices for rendering processor units-genes hybrid-rendered logistics for rendering, tracking, trending, effecting and controlling said networked repositories-rendered transactions and physical movements and locations of diverse industry-, industrial sector- and industrial sector categories-specific commodities and services and related financial transactions, said networked devices comprising networked repositories for optimizing people's health, healthcare, standard of living, tangible and intangible values, costs, prices, lifespans and evolving human gene pools, said networked repositories-rendered, updated and expanded relations of compilations of relations of individual groups of networked repositories-rendered, updated and expanded diverse industries-, industrial sectors- and categories-specific continuums of commodities and services related to said networked repositories-rendered, updated and expanded compilations of relations of digital diagnosis-specific snippets of relational chronologically ordered health and healthcare data and related relational profile data, comprising users' biometrics, demographics and professions data and device identifications, in the relational databases henceforth collectively known as said networked repositories-rendered, updated and expanded Abiotic Intelligence-Rendered digital ledgers for transacting, accounting and tallying said transactions of financials in national currencies and digital currencies including fiat currency and cryptocurrencies, and related transactions, trades, exchanges, barters and auctions of said continuums in said relational databases in said Abiotic Intelligence-Rendered digital ledgers henceforth known as AIR Pay wherein said individual groups of networked repositories for tracking, trending and effecting said individual groups of networked repositories-specific relations of compilations of relations in said AIR Pay based on said individual groups of networked repositories-rendered compilations of common relations of digital diagnosis-specific snippets, related continuums of commodities and services and related relational profile data henceforth known as AIR Pays, said Compilations of relations of snippets comprising inherent relational data for first rendering and serving as individual unique real-time digital tokens for authenticating, affirming, guaranteeing, safeguarding and transacting said transactions between and among said networked repositories, for second verifying, authenticating and affirming individual changes, modifications and updates in said relational profile data relating to individual networked repositories and individual networked repositories in said individual groups of networked repositories, and for third rendering inherent adaptable and versatile real-time system security for protecting and safeguarding said transactions, AIR Pay, AIR Pays collectively known as chainlinks in, between and among said networked repositories wherein said compilations of relations of said digital diagnosis-specific snippets including related relational profile data are the custodians and guarantors of said national currencies and digital currencies including fiat currency and cryptocurrencies in said transactions, wherein said individual groups of networked repositories for rendering current and projected comparative data on said financial transactions and related transactions and commerce in said compilations of relations and rendering said interactive dynamic real-time multi-dimensional visual hierarchied Comparative Values Prices Costs Ledgers for rendering and displaying said current and projected comparative data in the touchscreens and viewing screens of and among said networked devices, and said individual groups of networked repositories for rendering real-time statistical and actuarial analyses of said transactions and related analyses of financial transactions, usefulness, indications and cost-effectiveness of commodities, services and current and projected continuums and rendering interactive dynamic real-time multi-dimensional visual hierarchied Degree Distribution Graphs for rendering and displaying said real-time statistical and actuarial analyses and related abiotic intelligence-rendered findings, outcomes and products in said touchscreens and viewing screens, said Ledgers and graphs for rendering, tracking, trending, effecting and controlling said trades, exchanges, barters and auctions of said commodities and services and related financial transactions relating to said relational profile data in and based on national currencies, digital currencies including fiat currencies and cryptocurrencies among said individual groups of clients and purveyors networked repositories, said individual groups of networked repositories for applying said outcomes, products of said analytics as feedbacks and adjustments of said tangible and intangible values, prices and costs of said commodities, services and useful and indicated commodities, services in said continuums and related financial transactions in said compilations of relations of digital snippets-specific continuums in, between and among said networked repositories and individual groups of networked repositories, and applying said outcomes, products of said analytics as feedbacks and adjustments of said tangible and intangible values, prices and costs of said commodities, services and useful and indicated continuums and related trades, exchanges, barters and auctions among said individual groups of clients and purveyors networked repositories, said networked repositories comprise innovative computer programs including the innovative thesaurus software for attaching to and instructing processor units attached to memories in and among said networked repositories and individual groups of networked repositories wherein said processor units for rendering a plurality of electronic platforms for rendering, updating and expanding said AIR Pay, AIR Pays and chainlinks and for rendering a plurality of interactive digital visual-archied templates for rendering said platforms-rendered, updated and expanded compilations of relations in AIR Pay, AIR Pays and chainlinks in the touchscreens and viewing screens of said networked devices, said digital templates for rendering, updating and expanding said relational data in said relational databases according to said electronic platforms, said processor units-genes hybrid-rendered system comprising:
said networked repositories for rendering and updating said biometrics and demographics and professions data on physicians, patients, clients, purveyors, companies, corporations and third parties and networked device identifications known as relational profile data;
said individual groups of networked repositories for rendering, updating and expanding said individual groups-specific relational profile data in said individual groups-specific AIR Pays based on common relations of digital diagnosis-specific snippets in said relations of compilations of relations, said processor units for rendering a plurality of interactive digital visual hierarchied templates for rendering said individual groups-specific relational profile data in the touchscreens and viewing screens of said networked devices;
said networked repositories for rendering, updating and expanding said individual networked repositories-rendered AIR Pays, and AIR Pay and chainlinks;
said individual groups of networked repositories for rendering updating and expanding said individual groups of networked repositories-specific AIR Pays, and AIR Pay and chainlinks;
adaptable security, and interactive live and real-time Ledgers and Graphs for optimizing people's health and standard of living at low costs, altering and lengthening lifespans, evolving human gene pools associating with said individual and individual groups, wherein said individual compilations a said relations of digital snippets for rendering, successively updating and expanding the digital frameworks rendering and supporting said financial transactions in national currencies, digital currencies including fiat currency and cryptocurrencies for settling physical and digital debts, liabilities, credits and assets and for guaranteeing debts settlements and payments in said currencies and digital currencies including fiat currencies and cryptocurrencies in said transactions and e-commerce in said ledgers rendered by and among said networked repositories, networks of said networked devices comprising said individual networked repositories and individual groups of individual networked repositories including clients networked repositories, purveyors networked repositories, and clients and purveyors networked repositories comprising said processor units and said memories for storing said innovative computer programs, said relational databases including said relations of compilations of relations and successive compilations in said AIR Pay, AIR Pays and chainlinks and related products and outcomes of said Ledgers and Graphs;
networks of said networked devices comprising networked servers, mobile communication devices and desktops, said networked repositories comprising individual clients networked repositories, individual purveyors networked repositories, said networked repositories for rendering, updating and expanding said individual groups of clients and purveyors networked repositories-rendered, updated and expanded AIR Pay and AIR Pays wherein said individual groups of clients and purveyors networked repositories for rendering, updating and expanding said compilations of common relations of snippets and related said financial transactions, transactions of commodities, services and continuums, said networked repositories comprising said processor units attached said networked devices comprising said touchscreens and viewing screens for rendering and displaying said a plurality of interactive electronic industry-, industrial sector- and categories-specific platforms, said compilations of relations of digital snippets, AIR Pay and individual groups of networked repositories-specific AIR Pays, interactive real-time Ledgers and Graphs and edited and adaptable individual groups of networked repositories-specific AIR Pays;
said networked repositories for rendering a plurality of interactive electronic industries-, industrial sectors- and categories-specific platforms for rendering, updating and expanding said relations of compilations of relations of digital diagnosis-specific snippets of relational chronologically ordered medical and surgical chronologically ordered health and healthcare data and related industry-, industrial sector- and categories-specific commodities, services and continuums of said commodities and services and related financial transactions and related relational profile data in, between and among said networked repositories and individual groups of networked repositories;
said relations of compilations of relations of snippets for rendering peer-to-peer, device-to-device, devices-to-devices and device-to-servers transactions of AIR Pays in, between and among clients and purveyors networked repositories, said individual groups of networked repositories-rendered, -updated and -expanded said relations of compilations of relations of snippets for rendering adaptable and versatile real-time security for protecting said transactions, AIR Pay, AIR Pays, and chainlinks in, between and among said networked repositories from identities thefts, frauds, cyberattacks, hackings and corruptions, said compilations of relations of snippets are inherent in said relational databases wherein said compilations of relations of snippets for rendering said networked repositories-specific custodies and guarantees of said national currencies and digital currencies including fiat currencies and cryptocurrencies;
said individual groups of networked repositories for rendering, updating and expanding said individual groups of networked repositories-rendered, updated and expanded AIR Pays for tracking, trending and effecting said individual groups of networked repositories-specific AIR Pays in said AIR Pay and chainlinks;
said individual groups of networked repositories for rendering said interactive dynamic real-time multi-dimensional visual hierarchied Comparative Values Prices Costs Ledgers and associated interactive dynamic real-time multi-dimensional visual hierarchied Degree Distribution Graphs;
said networked devices comprising a plurality of commercial software for rendering people and users' biometrics data such as, but not limited to, facial including selfies biometrics, retina biometrics, voice biometrics, fingerprints, genetic fingerprints, amino acid sequences in DNA sequences, laboratory results of body fluids and tissues, sequences of binary numbers coding said biometrics data and laboratory images such as, digital x-ray, MRI, CTScan and ultrasound collectively known as laboratory tests; and
a plurality of near-field technologies for rendering and successively expanding and updating said relational databases, chainlinks and security in, between and among said networked repositories, a plurality of software Apps for rendering said system in and among said networked repositories.

2. The processor units-genes hybrid-rendered system according to claim 1 comprises said networked communication devices comprising said networked repositories and individual groups of networked repositories possessing processor units and attached memories for storing said innovative computer programs for hybridrizing said process units-rendered continuumns of commodities and services with said genes-rendered compilations of relations of digital snippets by said innovative computer programs instructing said processor units for said hybridrizations for tracking, trending, effecting and controlling said individual groups of networked repositories-rendered and -updated logistics for rendering and controlling physical movements and locations and transactions of diverse industry-, industrial sector- and industrial sector categories-specific commodities and services and related financial transactions, said individual groups of networked repositories-specific AIR Pays, individual groups of networked repositories-rendered, updated and expanded AIR Pay, Ledgers and Graphs, digital tokens and security and chainlinks.

3. The system according to claim 1 wherein said innovative compute programs including the innovative thesaurus software the innovative computer programs for instructing, said processor units and attached memories of said networked repositories and individual groups of networked repositories wherein said processor units for:
a. rendering an electronic platform for rendering and updating said biometrics and demographics and professions data on physicians, patients, clients, purveyors, companies, corporations and third parties and networked device identifications henceforth known as relational profile data, storing said relational data in said memories;
b. rendering said relational profile data in said continuums of diverse industry-, industrial sector- and categories-specific commodities and services as said relational profile data in, between and among said touchscreens and viewing screens of said networked devices and individual groups of networked devices;
c. rendering said individual networked repositories-rendered, -updated and -expanded and individual groups of networked repositories-rendered, -updated and -expanded compositions of relations of digital snippets, said individual groups for rendering and successively updating and expanding said compilations of relations of digital snippets relating to said relational data on said financial transactions and related continuums in said relational databases in said AIR Pay, said AIR Pays and chainlinks, adaptable security, and interactive live and real-time Ledgers and Graphs for optimizing people's health and standard of living at low costs, altering and lengthening lifespans, evolving human gene pools associating with said individual and individual groups, wherein said individual compilations of said relations of digital snippets for rendering, successively updating and expanding the digital frameworks rendering and supporting said financial transactions in national currencies, digital currencies including fiat currency and cryptocurrencies for settling physical and digital debts, liabilities, credits and assets and for guaranteeing debts settlements and payments in said currencies and digital currencies in said transactions and e-commerce in said ledgers rendered by and among said networked repositories;
d. applying commercial statistical and actuarial software for rendering said instant and real-time statistical and actuarial analyses of said transactions of said financials, commodities, services and current and projected continuums related and linked to said individual groups of networked repositories-rendered, updated and expanded compilations of relations of digital diagnosis-specific snippets of relational chronologically ordered health and healthcare data and related relational profile data in the dynamic updating and expanding compilations of said relational data in the relational databases in, between and among the memories of said networked repositories
e. rendering said individual groups of networked repositories-rendered, -updated and -expanded compilations, of relations of digital diagnosis-specific snippets for rendering adaptable and versatile real-time security for protecting said transactions, AIR Pay, Air Pays and chainlinks in, between and among said networked repositories from identities thefts, frauds, cyberattacks, backings and corruptions;
f. rendering said compilations of relations of digital diagnosis-specific snippets for rendering peer-to-peer, device-to-device, devices-to-devices and device-to-servers transactions of AIR Pays in, between and among said clients and purveyors networked repositories;
g. rendering said individual groups of networked repositories-rendered, -updated and -expanded compilations of relations of digital diagnosis-specific snippets for verifying individual changes, modifications and updates in said device identifications of individual networked devices in and among said individual groups of networked repositories in said relational databases in said chainlinks based on said processor units matching and verifying entire digital diagnosis-specific snippets in said individual subject networked repositories having said individual changes, modifications and updates within said compilations of relations of digital diagnosis-specific snippets in said individual groups of networked repositories-rendered AIR Pays whereby said innovative computer programs for instructing said processor units to relate said individual subject networked repositories to said compilations of relations of digital diagnosis-specific snippets in said memories;
h. rendering said compilations of relations of snippets for rendering said networked repositories-specific custodians and guarantors of said national currencies and digital currencies;
i. the thesaurus software for networking said networked repositories for rendering and successively expanding and updating said relational databases, compilations, AIR Pay, Air Pays and chainlinks, said networked repositories for rendering said chainlinks based on said compositions of relations of digital snippets comprising medical and surgical specialty-specific diagnoses and related and linked diagnoses-specific relational categories of chronologically ordered words, terms, terminologies and speeches in laypeople's languages and professionals' languages, images and videos;
j. attaching, updating and adjusting said tangible and intangible values, prices and costs in said national currencies and digital currencies of said commodities and services and said individual networked repositories- and individual groups of networked repositories-specific physical and digital debts, liabilities, credits and assets and guarantees and promissories for debts settlements and payments, said currencies and digital currencies including individual unique digital currencies keys, said processor units of said networked repositories for processing said submissions, updates into the relational databases in memories in, between and among said networked devices;
k. successively assigning, rendering, updating said tangible and intangible values, costs and prices in said currencies and digital currencies relating to said commodities and services and continuums;
l. rendering interactive dynamic real-time Comparative Values Prices Costs Ledgers to which attached said dynamic real-time Degree Distribution Graphs in the touchscreens and viewing screens of, between and among networked devices, said Comparative Values Prices Costs Ledgers for rendering streaming, live and instant current and projected comparative data on said individual groups of networked repositories-specific financial transactions and associated transactions and e-commerce of said commodities, services and continuums in said AIR Pays, AIR Pay, said digital tokens and security and chainlinks and said Degree Distribution Graphs for rendering streaming, live and instant statistical and actuarial analyses of said relational data and the products, outcomes and consequences of said Ledgers, said Ledgers and Graphs for optimizing people's health, healthcare, standard of living, tangible and intangible values, costs, prices, lifespans and evolving human gene pools in, between and among said touchscreens and viewing screens of and among said networked devices, wherein said values encompass monetary, qualitative and intangible values of said commodities and services;
m. processing said Ledgers-rendered and Graphs-rendered relational data as feedbacks and adjustments on said intangible and tangible values, prices and costs of said commodities, services and related digital snippets-specific useful and indicated commodities, services and continuums and related financial transactions including trades, exchanges, barters and auctions in AIR Pays and AIR Pay;
n. said interactive dynamic real-time Comparative Values Prices Costs Ledgers and in the attached dynamic real-time Degree Distribution Graphs for rendering, updating and expanding successive networked repositories-rendered transactions including trades, exchanges, barters and auctions wherein said interactive real-time Ledgers and Graphs for automatically rendering feedbacks on and adjusting said intangible and tangible values, prices and costs in said national currencies and digital currencies of said commodities, services and continuums and related financial transactions in said relational databases in said individual networked repositories- and individual groups of networked repositories-specific AIR Pays in AIR Pay;
o. applying said outcomes, products of said analytics as feedbacks and adjustments of said tangible and intangible values, prices and costs of said commodities, services and useful and indicated continuums and related financial transactions in said compilations of relations of digital snippets-specific continuums in, between and among said individual networked repositories and individual groups of networked repositories;
p. applying said outcomes, products of said analytics as feedbacks and adjustments of said tangible and intangible values, prices and costs of said commodities, services and useful and indicated continuums and related trades, exchanges, barters and auctions among said individual groups of clients and purveyors networked repositories;
q. calculating, accounting and tallying said financial transactions, accountings, transfers, payments, exchanges, barters, auctions, rendered feedbacks and analytics in, between and among said networked repositories-rendered AIR Pays in AIR Pay;
r. said compilations of relations of digital snippets, AIR Pay, AIR Pays, interactive real-time Ledgers and Graphs and chainlinks for optimizing people's health, healthcare, standard of living, appropriate and lower values, costs and prices for related useful commodities, services and continuums, lifespans and human gene pools associating with said individual networked repositories and individual groups of networked repositories for global commerce and socioeconomic engineering based on said technologies; and
s. rendering, streaming and displaying interactive real-time AIR Pays, relations of digital snippets, continuums and products and outcomes in interactive real-time electronic multi-dimensional visual-hierarchied ledgers and graphs said touchscreens and viewing screens of said networked repositories;
t. rendering a plurality of interactive digital templates for rendering said platforms-rendered, updated and expanded relational databases in the touchscreens and viewing screens of said networked devices, said digital templates for rendering, updating and expanding said relational data in said relational databases according to said electronic platforms.

4. The system according to claim 1 wherein said relations of compilations of relations whose fundamental structures in said relational databases in AIR Pays, AIR Pay, said digital tokens and security and chainlinks, said digital tokens and security and chainlinks comprise:
a representative relational profile data wherein biometrics data [*UB_data_p+n], [*UB_data_n+n] and [*UB_data_q+n] are almost always constant:
*unique key_p+n<>*unique key_n+n[created *TIMESTAMP_s]
*unique key_p+n<>*unique key_q+n[created *TIMESTAMP_n]
*unique key_q+n<>*unique key_q+n[created *TIMESTAMP_m]
Said networked repositories-related physicians' and purveyors' biometrics data and device and demographics data:
*unique key_p+1=[*physician_p+1_data]<[*UB_data_p+1]>[created *TIMESTAMP_a+1]
*unique key_p+1=[*physician_p+1_data]<[*UB_data_p+1]>[created *TIMESTAMP_a+2]
*unique key_p+2=[*physician_p+2_data]<[*UB_data_p+2]>[created *TIMESTAMP_a+4]
*unique key_p+n=[*physician_p+n_data]<[*UB_data_p+n]>[created *TIMESTAMP_a+n]
*unique key_p+1<[*UD_data_y+1]>[created *TIMESTAMP_a+1]
*unique key_p+1<[*UD_data_y+1]>[modified *TIMESTAMP_a+2]
*unique key_p+2<[*UD_data_y+2]>[modified *TIMESTAMP_a+4]
*unique key_p+n<[*UD_data_y+n]>[created *TIMESTAMP_a+n]
Said networked repositories-related patients' and clients' biometrics data and device and demographics data:
*unique key_n+1=[*patient_n+1_data]<[*UB_data_n+1]>[created *TIMESTAMP_a+6]
*unique key_p+1=[*patient_n+1_data]<[*UB_data_n+1]>[modified *TIMESTAMP_a+11]
*unique key n+n=[*patient_n+_data]<[*UB_data_n+n]>[created *TIMESTAMP_a+n]
*unique key_n+1<[*UD_data_y+10]>[created *TIMESTAMP_a+6]
*unique key_n+1<[*UD_data_y+133]>[modified *TIMESTAMP_a+11]
*unique key_n+n<[*UD_data_y+4536]>[created *TIMESTAMP_a+n]
Said networked repositories-related clients' and purveyors' biometrics data and device and demographics data:
*unique key_q+1=[*user_q+1_data]<[*UB_data_q+1]>[created *TIMESTAMP_a+1]
*unique key_q+1=[*user_q+1_data]<[*UB_data_q+1]>[modified *TIMESTAMP_a+3]
*unique key_q+2=[*user_q+2_data]<[*UB_data_q+2]>[created *TIMESTAMP_a+2]
*unique key_q+n=[*user_q+n_data]<[*UB_data_q+n]>[created *TIMESTAMP_a+n]
*unique key_q+1<[*UD_data_y+1]>[created *TIMESTAMP_a+1]
*unique key_q+1<[*UD_data_y+3]>[modified *TIMESTAMP_a+3]
*unique key_q+2<[*UD_data_y+2]>[created *TIMESTAMP_a+2]
*unique key_q+n<[*UD_data_y+5]>[created *TIMESTAMP_a+n]
Individual networked repositories-rendered, updated and expanded and individual groups of networked repositories-rendered, updated and expanded digital diagnosis-specific snippets of relational chronologically ordered health and healthcare data eluding values prices and costs and related relational profile data, accounting and tally functionalities are not shown here:
*unique key*_p+1<[*foreign key_m+1][*foreign key_m+2][*foreign key_m+3][created *TIMESTAMP_a+3]
*unique key*_p+1<[*foreign key_m+4][*foreign key_m+5][created *TIMESTAMP_a+111]
*unique key*_p+1<[*foreign key_m+n][created *TIMESTAMP_a+n]
*unique key*_n+1<[*foreign key_m+1][*foreign key_m+2][*foreign key_m+3][created *TIMESTAMP_a+14]
*unique key*_n+1<[*foreign key_m+4][*foreign key_m+5][created *TIMESTAMP_a+111]
*unique key*_n+1<[*foreign key_m+n][created *TIMESTAMP_a+n+11]
*unique key*_n+1<[*foreign key_o+1][*foreign key_o+2][*foreign key_o+3][created *TIMESTAMP_a+14]
*unique key*_n+1<[*foreign key_o+4][*foreign key_o+5][created *TIMESTAMP_a+111]
*unique key*_n+1<[*foreign key_o+n][created *TIMESTAMP_a+n+11]
*unique key_m+1<*diagnose_x+1_data<*RCOHH_data_x+1>[created *TIMESTAMP_a+2]
*unique key_m+2<diagnose_x+2_data<*RCOHH_data_x+2>[created *TIMESTAMP_a+3]
*unique key_m+3<*diagnose_x+3_data<*RCOOH_data_x+3>[created *TIMESTAMP_a+4]
*unique key_m+4<*diagnose_x+4_data<*RCOHH_data_x+4>[created *TIMESTAMP_a+5]
*unique key_m+5<*diagnose_x+5_data<*RCOHH_data_x+5>[created *TIMESTAMP_a+6]
*unique key_m+n<*diagnose_x+n_data<*RCOHH_data_x+n>[created *TIMESTAMP_a+n]
[*foreign key_m+1]=*unique key_m+1<*diagnose_x+1_data<*RCOHH_data_x+1>[created *TIMESTAMP_a+2]
[*foreign key_m+2]=*unique key_m+2<*diagnose_x+2_data<*RCOHH_data_x+2>[created *TIMESTAMP_a+3]
[*foreign key_m+3]=*unique key_m+3<*diagnose_x+3_data<*RCOHH_data_x+3>[created *TIMESTAMP_a+4]
[*foreign key_m°4]=*unique key_m+4<*diagnose_x+4_data<*RCOHH_data_x+4>[created *TIMESTAMP_a+5]
[*foreign key_m+5]=*unique key_m+5<*diagnose_x+5_data<*RCOHH_data_x+5>[created *TIMESTAMP_a+6]
[*foreign key_m+n]=*unique key_m+n<*diagnose_x+n_data<*RCOHH_data_x+n>[created *TIMESTAMP_a+n]
[*foreign key_o+1]=*unique key_o+1<*STTPDEP_data_x+1<[*values_data_o+1]
[*prices_data_o+1][*costs_data_o+1]>[created *TIMESTAMP_a+2]
[*foreign key_o+2]=*unique key_o+2<*STTPDEP_data_x+2<[*values_data_o+2]
[*prices_data_o+2][*costs_data_o+2]>[created *TIMESTAMP_a+3]
[*foreign key_o+3]=*unique key_o+3<*STTPDEP_data_x+3<[*values_data_o+3]
[*prices_data_o+3][*costs_data_o+3]>[created *TIMESTAMP_a+4]
[*foreign key_o+4]=*unique key_o+4<*STTPDEP_data_x+4<[*values_data_o+4]
[*prices_data_o+4][*costs_data_o+4]>[created *TIMESTAMP_a+5]
[*foreign key_o+5]=*unique key_o+5<*STTPDEP_data_x+5<[*values_data_o+5]
[*prices_data_o+5][*costs_data_o+5]>[created *TIMESTAMP_a+6]
[*foreign key_o+n]=*unique key_o+n<*STTPDEP_data_x+n<[*values_data_o+n]
[*prices_data_o+n][*costs_data_o+n]>[created *TIMESTAMP_a+n]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1],
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_p+1<[*foreign key_o+1]>[created *TIMESTAMP_a+2]
*unique key_p+1<[*foreign key_o+n]>[created *TIMESTAMP_a+5]
*unique key_p+n<[*foreign key_o+n]>[created *TIMESTAMP_a+6]
*unique key_n+1<[*foreign key_o+1]>[created *TIMESTAMP_a+2]
*unique key_n+1<[*foreign key_o+n]>[created *TIMESTAMP_a+5]
*unique key_n+n<[*foreign key_o+n]>[created *TIMESTAMP_a+6]
Individual networked repositories-rendered, updated and expanded and individual networked repositories-rendered, updated and expanded commodities and services including, values prices and costs:
*unique key_m+1<[*foreign key_o+1]>[created *TIMESTAMP_a+2]
*unique key_m+2<[*foreign key_o+2]>[created *TIMESTAMP_a+3]
*unique key_m+3<[*foreign key_o+3]>[created *TIMESTAMP_a+4]
*unique key_m+4<[*foreign key_o+4]>[created *TIMESTAMP_a+5]
*unique key_m+5<[*foreign key_o+5]>[created *TIMESTAMP_a+6]
*unique key_m+n<[*foreign key_o+n]>[created *TIMESTAMP_a+n]
*unique key*_q+1<[*foreign key_f+1]>[created *TIMESTAMP_c+2]
*unique key*_q+2<[*foreign key_f+2]>[created *TIMESTAMP_c+3]
*unique key*_q+1<[*foreign key_f+3]>[created *TIMESTAMP_c+4]
*unique key_m+4<[*foreign key_f+4]>[created *TIMESTAMP_c+5]
*unique key_m+5<[*foreign key_f+5]>[created *TIMESTAMP_c+6]
*unique key_m+n<[*foreign key_f+n]>[created *TIMESTAMP_c+n]
*unique key_q+1=[*user_n+1_data]<[*UB_data_x+1]<[*UD_data_y+1]>[created *TIMESTAMP_g+1]
*unique key_q+1=[*user_n+1_data]<[*UB_data_x+1]<[*UD_data_y+2]<[modified *TIMESTAMP_g+2]
*unique key_q+1=[*user_n+1_data]<[*UB_data_x+1]<[*UP_data_y+3]<[modified *TIMESTAMP_g+n]
*unique key_q+2=[*user_n+1_data]<[*UB_data_x+1]<[*UD_data_y+1]>[created *TIMESTAMP_g+4]
*unique key_q+3=[*user_n+1_data]<[*UB_data_x+1]<[*UD_data_y+1]>[created *TIMESTAMP_g+4]
*unique key_q+4=[*user_n+1_data]<[*UB_data_x+1]<[*UD_data_y+1]>[created *TIMESTAMP_g+8]
*unique key_q+n=[*user_n+1_data]<[*UB_data_x+1]<[*UD_data_y+1]>[created *TIMESTAMP_g+11]
*unique key_q+1<[*foreign key_e+1][created *TIMESTAMP_a+3]
*unique key q+2<[*foreign key_e+4][created *TIMESTAMP_a+111]
*unique key_q+3<[*foreign key_e+n][created *TIMESTAMP_a+166]
*unique key_q+4<[*foreign key_e+n][created *TIMESTAMP_a+166]
*unique key_q+n<[*foreign key_e+n][created *TIMESTAMP_a+12]
[*foreign key_e+1]=*unique key_e+1<*commodities_services_data_x+1<[**values_data_x+1][**prices_data_x+1][**costs_data_x+1]>[created *TIMESTAMP_a+2]
[*foreign key_e+2]=*unique key_e+2<*commodities_services_data_x+2<[**values_data_x+2][**prices_data_x+2][**costs_data_x+2]>[created *TIMESTAMP_a+3]
[*foreign key_e+3]=*unique key_e+3<*commodities_services_data_x+3<[**values_data_x+3][**prices_data_x+3][**costs_data_x+3]>[created *TIMESTAMP_a+4]
[*foreign key_e+4]=*unique key_e+4<*commodities_services_data_x+4<[**values_data_x+4][**prices_data_x+4][**costs_data_x+4]>[created *TIMESTAMP_a+5]
[*foreign key_e+5]=*unique key_e+5<*commodities_services_data_x+5<[**values_data_x+5][**prices_data_x+5][**costs_data_x+5]>[created *TIMESTAMP_a+6]
[*foreign key_e+6]=*unique key_e+6<*commodities_services_data_x+6<[**values_data_x+6][**prices_data_x+6][**costs_data_x+6]>[created *TIMESTAMP_a+7]
[*foreign key_e+n]=*unique key_e+n<*commodities_services_data_x+n<[**values_data_x+n][**prices_data_x+n][**costs_data_x+n]>[created *TIMESTAMP_a+n]
wherein said *commodities_services_data_x+n represent commodities and services in, but not limited to, health and healthcare commodities and services, devices and equipment, pharmaceuticals, food and beverages, financials, investment, estate planning and insurance, actuarial services, community and social services, acute, chronic, long-term and end-of-life care, legal services, communication, real estate, travel and transportation, hotel and hospitality industries;
Individual groups of networked repositories-rendered, updated and expanded diverse digital snippets-specific continuums of commodities and services and continuums related to compilations of relations of digital diagnosis-specific snippets of relational chronologically ordered health and healthcare data and related relational profile data, said compilations of common relations of digital diagnosis-specific snippets for rendering said individual groups of networked repositories:
*unique key_n+1=[*patient_n+1_data]<[*UB_data_n+1]<[*UD_data_y+10]>[created *TIMESTAMP_a+6]
*unique key_n+1[*patient_n+1_data]<[*UB_data_n+1]<[*UD_data_y+133]<[modified *TIMESTAMP_a+11]
*unique key_n+n=[*patient_n+n_data]<[*UB_data_n+n]<[*UD_data_y+4536]>[created *TIMESTAMP_a+n]
*unique key_n+1<[*foreign key_m+1]<[*foreign key_e+1][created *TIMESTAMP_a+2]
*unique key_n+1<[*foreign key_m+1]<[*foreign key_e+2][created *TIMESTAMP_a+4]
*unique key_n+1<[*foreign key_m+1]<[*foreign key_e+3][created *TIMESTAMP_a+6]
*unique key_n+1<[*foreign key_m+1]<[*foreign key_e+4][created *TIMESTAMP_a+8]
*unique key_n+1<[*foreign key_m+1]<[*foreign key_e+n][created *TIMESTAMP_a+10]
*unique key_n+1<[*foreign key_m+n]<[*foreign key_e+n][created *TIMESTAMP_a+n]
*unique key_n+n<[*foreign key_m+1]<[*foreign key_e+1][created *TIMESTAMP_a+3]
*unique key_n+n<[*foreign key_m+5]<[*foreign key_e+2][created *TIMESTAMP_a+5]
*unique key_n+n<[*foreign key_m+5]<[*foreign key_e+4][created *TIMESTAMP_a+7]
*unique key_n+n<[*foreign key_m+5]<[*foreign key_e+6][created *TIMESTAMP_a+9]
*unique key_n+n<[*foreign key_m+5]<[*foreign key_e+n][created *TIMESTAMP_a+n+1]
wherein a representative compilation of common relations of digital diagnosis-specific snippets of relational chronologically ordered health and healthcare data and related relational profile data are schematically represented by a common [*foreign key_m+1], said individual groups of networked repositories for rendering, updating and expanding AIR Pays, AIR Pay, said digital tokens and security and chainlinks based on common relations of digital diagnosis-specific snippets in said relations of compilations of relations:
*unique key_n+1<[*foreign key_m+1]<[*foreign key_e+1][created *TIMESTAMP_a+2]
*unique key_n+n<[*foreign key_m+1]<[*foreign key_e+1][created *TIMESTAMP_a+3]
where said *user represent people and entities associating with said networked devices such as clients, purveyor, physicians, patients, payees, payers, companies, corporations, third parties, etc. Said a, c, e, f, g, n, m, o, p, q, r, s, x, y, z each of which represents 0 people, 0 user or 0 commodities and services and 1, 2, 3, 4 . . . infinite number of people users or commodities and services; all people become patients and clients and some people are physicians and purveyors; “*” and “**” represent “a, each, every”, *UB_data represent a, each, every or all unique biometrics data of people and users, *UD_data represent a, each, every or all device data and demographics data of people and users; *RCOHH_data represents diagnosis*-specific chronologically ordered health and healthcare relational data; *STTPDEP represents a plurality of office Visit and consultation services, laboratory tests, treatments, procedures, devices, equipments, pharmaceuticals and others some of which are associated with said *values, *costs and *prices; and same concept applied to said commodities, services and continuums associated with said **values, **costs, and **prices; categories, subcategories, headings or headers, topics or types in medicine, surgeries and health-care as described in the preferred embodiments specialty*- and diagnosis*-specific Drug Allergy, Food Allergy, Chief Complaints, Symptoms, signs, laboratory tests, diagnoses, differential diagnoses, treatments, procedures, pharmaceuticals, diverse levels of office Visit and consultations, recommendations, referrals, hospitals, Clinics, notes, messages; said [*values] [*prices] [*costs] [**values] [**prices] [**costs] of said commodities and services and continuums in said financial transactions, accountings, transfers, payments and credits are transacted in national currencies, digital currencies including fiat currency and cryptocurrencies; said symbols are arbritary and may not conform to the standard computer programming languages.

5. The compilations of relations of digital diagnosis-specific snippets of relational chronologically ordered health and healthcare data according to claim 4 comprise medical and surgical specialties-specific diagnoses and diagnoses-specific relational categories of diverse clinical and hospital office Visit and consultation services, chief Complaints, symptoms, signs, laboratory tests, pharmaceuticals, diagnoses, related and linked diagnoses, differential diagnoses, treatments, procedures, devices, equipments, recommendations, referrals, hospitals, clinical notes and messages, drug allergy, food allergy, wherein said diagnoses-specific chief Complaints, symptoms, signs and treatments are expressed in chronologically ordered words, terms, terminologies, images, videos and speeches based on the actual chronologically expressions of diseases and disorders, said chronologically ordered words, terms, terminologies and speeches are in laypeople's languages and professionals' languages; and
individual clinical and hospital office Visit and consultation services, laboratory tests, pharmaceuticals, treatments, procedures, devices and equipments are associated with individual values, prices and costs in said national currencies, digital currencies eluding fiat currency and cryptocurrencies in said memories in, between and among said networked repositories.

6. The system according to claim 1 wherein said Comparative Values Prices Costs Ledgers and Degree Distribution Graphs for rendering, tracking, trending, effecting and controlling said trades, exchanges, barters and auctions of said commodities and services and related financial transactions relating to said relational profile data among said individual groups of clients and purveyors networked repositories comprise said processor units for:
rendering interactive electronic platforms for rendering said interactive dynamic real-time Comparative Values Prices Costs Ledgers for rendering, updating and expanding said successive individual groups of networked repositories-rendered transactions including trades, exchanges, barters and auctions relating to comparative values, prices and costs and cost-effectiveness of said commodities, services and current and projected continuums and said dynamic real-time Degree Distribution Graphs for executing instant and real-time statistical and actuarial analyses of said transactions and analyses of financial transactions, usefulness, indications and cost-effectiveness of commodities, services and current and projected continuums in said relational databases;
applying said outcomes, products of said analytics as feedbacks and adjustments of said values, prices and costs of said commodities, services and useful and indicated commodities, services in said continuums and related financial transactions in said compilations of relations of digital snippets-specific continuums in said relational databases in and among individual networked repositories and individual groups of networked repositories; and
rendering a plurality of interactive electronic seal hierarchied templates for rendering and streaming said outcomes and products of said analytics, feedbacks and adjustments of said values, prices and costs of said commodities, services and useful and indicated continuums in said trades, exchanges, barters and auctions in said touchscreens and viewing screens of said individual groups of clients and purveyors networked devices.

7. The system according to claim 1 wherein said compilations of relations of snippets for rendering peer-to-peer, device-to-device, devices-to-devices and device-to-servers transactions of AIR Pays in, between and among clients and purveyors networked repositories, said individual groups of networked repositories-rendered, -updated and -expanded said compilations of relations of snippets for rendering adaptable and versatile real-time security for protecting said transactions, AIR Pay, AIR Pays, chainlinks and system in, between and among said networked repositories, said compilations of relations of snippets are inherent in said relational databases in said chainlinks wherein said compilations of relations of snippets for rendering said networked repositories-specific custodies and guarantees of said national currencies and digital currencies comprise:
Compilations of relations of snippets comprising inherent relational profile data for serving as individual real-time unique tokens for said transactions, inherent real-time security and custodians and guarantees:
*unique key_p+n<>*unique key_n+n[created *TIMESTAMP_s]
*unique key_p+n<>*unique key_q+n[created *TIMESTAMP_n]
*unique key_q+n<>*unique key_q+n[created *TIMESTAMP_m]
*unique key_p+n<[*foreign key_m+n][created *TIMESTAMP_a+n]
*unique key_n+n<[*foreign key_m+n][*foreign key_o+n][*foreign key_e+n][created *TIMESTAMP_a+n]
*unique key_q+n<[*foreign key_e+n][created *TIMESTAMP_a+n]

8. The system according to claim 1 wherein said Compilations of relations of snippets comprising inherent relational data for second verifying, authenticating and affirming individual changes, modifications and updates in said relational profile data comprise:
said relational profile data comprising individual changes, modifications and updates being integral relational data in said relations of compilations in said AIR Pays, AIR Pay, said digital tokens and security and chainlinks; and individual networked repositories-rendered individual constant biometrics data *unique key_p+n, *unique key_q+n and *unique key_q+n and individual groups of networked repositories-rendered individual groups of networked repositories-rendered, -updated and -expanded compilations of relations of snippets in said relational databases in said chainlinks:
A representative excerpt of said relational profile data in said relational databases in AIR Pays, AIR Pay, said digital tokens and security and chainlinks wherein biometrics data [*UB_data_p+n], [*UB_data_n+n] and [*UB_data_q+n] are constant while [*UD_data_y+n] [*UD_data_y+4536][*UD_Data_y+m] can be modified albeit verified and authenticated by said relational data biometrics data in said relational data in said relations of compilations in said AIR Pays, AIR Pay, said digital tokens and security and chainlinks:
*unique key_p+n<>*unique key_n+n[created *TIMESTAMP_s]
*unique key_p+n<>*unique key_q+n[created *TIMESTAMP_n]
*unique key_q+n<>*unique key_q+n[created *TIMESTAMP_m]
Said networked repositories-related physicians' and purveyors' biometrics data and device identification and demographics data:
*unique key_p+1=[*physician_p+1_data]<[*UB_data_p+1]>[created *TIMESTAMP_a+1]
*unique key_p+1=[*physician p+1_data]<[*UB_data_p+1]>[created *TIMESTAMP_a+2]
*unique key_p+2=[*physician_p+2_data]<[*UB_data_p+2]>[created *TIMESTAMP_a+4]
*unique key_p+n=[*physician_p+n_data]<[*UB_data_p+n]>[created *TIMESTAMP_a+n]
*unique key_p+1<[*UD_data_y+1]>[created *TIMESTAMP_a+1]
*unique key_p+1<[*UD_data_y+1]>[modified *TIMESTAMP_a+2]
*unique key_p+2<[*UD_data_y+2]>[modified *TIMESTAMP_a+4]
*unique key_p+n<[*UD_data_y+n]>[created *TIMESTAMP_a+n]
Said networked repositories--related patients' and clients' biometrics data and device identification and demographics data:
*unique key_n+1=[*patient_n+1_data]<[*UB_data_p+1]>[created *TIMESTAMP_a+6]
*unique key_n+1=[*patient_n+1_data]<[*UB_data_n+1]>[created *TIMESTAMP_a+11]
*unique key_n+n=[*patient_n+n_data]<[*UB_data_n+n]>[created *TIMESTAMP_a+n]
*unique key_n+1<[*UD_data_y+10]>[created *TIMESTAMP_a+6]
*unique key_n+1<[*UD_data_y+133]>[modified *TIMESTAMP_a+11]
*unique key_n+n<[*UD_data_y+4536]>[created *TIMESTAMP_a+n]
Said networked repositories-related clients' and purveyors' biometrics data and device identification and demographics data:
*unique key_q+1=[*user_q+1_data]<[*UB_data_q+1]>[created *TIMESTAMP_a+1]
*unique key_q+1=[*user_q+1_data]<[*UB_data_q+1]>[created *TIMESTAMP_a+3]
*unique key_q+2=[*user_q+2_data]<[*UB_data_q+2]>[created *TIMESTAMP_a+2]
*unique key_q+n=[*user_q+n_data]<[*UB_data_q+n]>[created *TIMESTAMP_a+n]
*unique key_q+1<[*UD_data_y+1]>[created *TIMESTAMP_a+1]
*unique key_q+1<[*UD_data_y+3]>[modified *TIMESTAMP_a+3]
*unique key_q+2<[*UD_data_y+2]>[created *TIMESTAMP_a+2]
*unique key_q+n<[*UD_data_y+m]>[created *TIMESTAMP_a+n]
*unique key*_p+1<[*foreign key_m+1][*foreign key_m+2][*foreign key_m+3][created *TIMESTAMP_a+3]
*unique key*_p+1<[*foreign key_m+4][*foreign key_m+5][created *TIMESTAMP_a+111]
*unique key*_p+1<[*foreign key_m+n][created *TIMESTAMP_a+n]
*unique key*_n+1<[*foreign key_m+1][*foreign key_m+2][*foreign key_m+3][created *TIMESTAMP_a+14]
*unique key*_n+1<[*foreign key_m+4][*foreign key_m+5][created *TIMESTAMP_a+11]
*unique key*_n+1<[*foreign key_m+n][created *TIMESTAMP_a+n+11]
*unique key*_n+1<[*foreign key_o+1][*foreign key _o+2][*foreign key_o+3][created *TIMESTAMP_a+14]
*unique key*_n+1<[*foreign key_o+4][*foreign key_o+5][created *TIMESTAMP_a+111]
*unique key*_n+1<[*foreign key_o+n][created *TIMESTAMP_a+n+11]
said processor units for matching, verifying and authenticating entire digital diagnosis-specific snippets in said individual subject networked repositories having said individual changes, modifications and updates within said compilations of relations of digital diagnosis-specific snippets in said individual groups of networked repositories-rendered AIR Pays whereby said innovative computer programs for instructing said processor units to relate said individual subject networked repositories to said compilations of relations of digital diagnosis-specific snippets in said memories.

9. In the processor units-genes-rendered hybrid system of networked repositories-rendered, updated and expanded Abiotic Intelligence-Rendered digital ledgers for tracking, trending, effecting and controlling said networked repositories for optimizing people's health, healthcare, standard of living, tangible and intangible values, costs, prices, lifespans and evolving human gene pools, said networked repositories-rendered, updated and expanded relations of compilations of relations of individual groups of networked repositories-rendered, updated and expanded diverse industries-, industrial sectors- and categories-specific continuums of commodities and services related to said networked repositories-rendered, updated and expanded compilations of relations of digital diagnosis-specific snippets of relational chronologically ordered health and healthcare data and related relational profile data, comprising users' biometrics, demographics and professions data and device identifications, in the relational databases henceforth collectively known as said networked repositories-rendered, updated and expanded Abiotic Intelligence-Rendered digital ledgers for transacting, accounting and tallying said transactions of financials in national currencies and digital currencies including fiat currency and cryptocurrencies, and related transactions, trades, exchanges, barters and auctions of said continuums in said relational databases in said Abiotic Intelligence-Rendered digital ledgers henceforth known as AIR Pay wherein said individual groups of networked repositories for tracking, trending and effecting said individual groups of networked repositories-specific relations of compilations of relations in said AIR Pay based on said individual groups of networked repositories-rendered compilations of common relations of digital diagnosis-specific snippets, related continuums of commodities and services and related relational profile data henceforth known as AIR Pays, said system comprising individual unique real-time digital tokens for authenticating, affirming, guaranteeing, safeguarding and transacting said transactions, for verifying, authenticating and affirming individual changes, modifications and updates in said atonal profile data, for rendering inherent adaptable and versatile real-time system security for rendering the custodians and guarantors of said national currencies and digital currencies including fiat currency and cryptocurrencies, Comparative Values Prices Costs Ledgers and related Degree Distribution Graphs for optimizing said values, prices and costs in said financial transactions relating to said usefulness, indications and cost-effectiveness of said commodifies, services and current and projected continuums and related trades, exchanges, barters and auctions among said networked repositories, the method of said individual groups of networked repositories rendering processor units-genes hybrid-rendered logistics rendering, tracking, trending, effecting and controlling said individual groups-rendered, tracked, trended, effected and controlled physical movements and locations and transactions of diverse industry-, industrial sector- and industrial sector categories-specific commodities and services and related accountings and tallies of financial transactions in and among said networked repositories, said method comprises process of:
networking said networked devices comprising networked servers, mobile communication devices and desktops, said networked repositories comprising individual client networked repositories and individual purveyors networked repositories, said networked repositories rendering, successively updating and expanding said individual groups of clients and purveyors networked repositories-rendered, updated and expanded AIR Pay and AIR Pays hereby said individual groups of clients and purveyors networked repositories rendering, updating and expanding said compilations of common relations of snippets and related said financial transactions, transactions of commodities, services and continuums, said networked repositories comprising said processor units and memories for rendering and storing said innovative computer programs, said relational databases including said compilations and successive compilations in said AIR Pay and related products and outcomes of said Ledgers and Graphs and chainlinks, said networked devices comprising said touchscreens and viewing screens rendering and displaying said a plurality of interactive electronic industry-, industrial sector- and categories-specific platforms, said compilations of relations and relations of compilation of digital snippets, said AIR Pay, and individual groups of networked repositories-specific AIR Pays, interactive real-time Ledgers and Graphs and edited and adaptable individual groups of networked repositories-specific AIR Pays;
said networked repositories having and using a plurality of commercial software rendering said users' biometrics data—facial including selfies biometrics, retina biometrics, voice biometrics, fingerprints, genetic fingerprints, amino acid sequences in DNA sequences, laboratory results of body fluids and tissues, sequences of binary numbers coding said biometrics data and laboratory images such as, digital, x-ray, MRI, CTScan and ultrasound;
said networked repositories having and using a plurality of near field technologies rendering and successively expanding and updating said relational databases, chair links and security in, between and among said networked repositories, a plurality of software Apps for rendering said system in and among said networked repositories;
said networked devices implementing a plurality of commercial software for rendering said users' biometrics data such as, but not limited to, facial including selfies biometrics, retina biometrics, voice biometrics, fingerprints, genetic fingerprints, amino acid sequences in DNA sequences, laboratory results of body fluids and tissues, sequences of binary numbers coding said biometrics data, and laboratory images such as, digital x-ray, MRI, CTScan and ultrasound;
a plurality of near field technologies for rendering and successively expanding and updating said relational databases, chainlinks and security in, between and among said networked repositories, and a plurality of software Apps for rendering said system in and among said networked repositories;
said networked repositories-rendered Abiotic Intelligence tracking, trending, effecting and controlling said relational databases to comprise compilations of relational and relations of compilations of relational financial transactions relating to said transactions and commerce of goods and services in diverse industries and industrial sectors henceforth commodities and services and said diverse continuums of commodities and services related to said networked repositories-rendered, updated and expanded compilations of relations of digital diagnosis-specific snippets of relational chronologically ordered health and healthcare data and related relational profile data in said relational databases whereby said individual groups of said networked repositories for rendering, updating and expanding said continuums to comprise relations of digital snippets-specific commodities, services and continuums being processed by said processor units and stored in said memories of said networked repositories henceforth collectively known as networked repositories-rendered Abiotic Intelligence-Rendered digital ledgers accounting and tallying said transactions of financials, commodities, services and continuums in, between and among clients and purveyors networked repositories henceforth known as AIR Pay;
whereby said networked repositories rendering, updating and expanding individual networked repositories-specific AIR Pays and individual groups of said individual networked repositories-specific AIR Pays comprising said compilations of common relations of digital diagnosis-specific snippets from said AIR Pay being rendered and viewed in and among the touchscreens and viewing screens of networked devices, and inherent in said AIR Pay and AIR Pays are said individual digital snippets and individual compilations of relations of said digital snippets rendering and safeguarding the custodians of said currencies and digital currencies in, between and among said networked repositories whereby said digital snippets rendering said transactions and rendering adaptable and versatile real-time security protecting said transactions, AIR Pay, AIR Pays, chainlinks and system in, between and among said networked repositories;
whereby said individual groups of individual networked repositories rendering, updating and expanding interactive dynamic real-time Comparative Values Prices Costs Ledgers to which attached said dynamic real-time Degree Distribution Graphs in the touchscreens and viewing screens of, between and among networked devices rendering, updating and expanding successive interactive and dynamic streaming real-time Comparative Values Prices Costs Ledgers rendering streaming, live and instant current and projected successive comparative data on said individual groups of networked repositories-specific financial transactions and associated transactions and e-commerce of said continuums in said AIR Pay, AIR Pays and successive diverse interactive and dynamic streaming real-time Degree Distribution Graphs rendering streaming, live and instant statistical and actuarial analyses of said data;
whereby said individual groups of networked repositories feedback and adjust said tangible and intangible values, prices and costs in said national currencies and digital currencies of said commodities, services and continuums and related financial transactions in said relational databases in said AIR Pay and AIR Pays based on said products, outcomes and consequences of said analyses relating to said individual groups of networked repositories-rendered compilations of relations and relations compilations of relations of snippets in said relational databases in said AIR Pay and AIR Pays by said processor units whereby said individual networked repositories- and individual groups of networked repositories-specific AIR Pays to optimize people's health, healthcare, standard of living, tangible and intangible values, costs, prices, lifespans and evolving human gene pools in, between and among said touchscreens and viewing screens of and among said networked devices, wherein said values encompass monetary, qualitative and intangible values of said commodities and services;
whereby said individual networked repositories and individual groups of individual networked repositories including clients and purveyors networked repositories rendering, updating and expanding said AIR Pay and AIR Pays therein said networked repositories for rendering a plurality of interactive electronic industry-, industrial sector- and categories-specific platforms for rendering, updating and expanding said compilations of relations of digital diagnosis-specific snippets of relational chronologically ordered medical, surgical, users' health and healthcare data and related relational profile data and related industry-, industrial sector- and categories-specific said commodities, services and said digital snippets-specific continuums of said commodities and services and related financial transactions, and wherein said individual groups and clients and purveyors for rendering a plurality of interactive electronic industry-, industrial sector- and categories-specific platforms for rendering, updating and expanding said compilations of common relations of digital diagnosis-specific snippets of relational chronologically ordered medical, surgical, users' health and healthcare data and related relational profile data and related industry-, industrial sector- and categories-specific said commodities, services and continuums and related financial transactions in, between and among said networked repositories wherein said continuums comprising said digital snippets-specific commodities and services;
whereby said compilations of relations of snippets rendering peer-to-peer, device-to-device, devices-to-devices and device-to-servers transactions in, between and among clients and purveyors networked repositories, said individual groups of networked repositories-rendered, -updated and -expanded said compilations of relations of snippets rendering adaptable and versatile real-time security protecting said transactions, AIR Pay, AIR Pays, chainlinks and system in, between and among said networked repositories from identities thefts, frauds, cyberattacks, hackings and corruptions, said compilations of relations of snippets are inherent in said relational databases in said chainlinks whereby said compilations of relations of snippets rendering and guaranteeing said networked repositories-specific custodians and guarantees of said national currencies and digital currencies;
said innovative computer programs instructing said processor units and attached memories of said networked repositories and individual groups of networked repositories to render, update and expand said relations of compilations of relations of digital snippets in said AIR Pay, AIR Pays in said AIR PAY, and how to calculate, perform, account and tally said financial transactions, accountings, transfers, payments, exchanges, barters and auctions in, between and among said networked repositories and said analytics by said Ledgers and Graphs and feedbacks in said AIR Pay and AIR Pays, said processor units and attached memories; and
said innovative computer programs instructing said processor units attached to said memories of said networked repositories and individual groups of networked repositories whereby said processor units:
a1. rendering an electronic platform for rendering and updating said biometrics and demographics data physicians, patients, clients, purveyors, companies, corporations and third parties and networked device identifications, storing said relational data in said memories;
a2. rendering said relational profile data in said continuums of diverse industry-, industrial sector- and categories-specific commodities and services as said relational profile data in, between and among said touchscreens and viewing screens of said networked devices and individual groups of networked devices;
b. rendering said individual networked repositories-rendered, -updated and -expanded and individual groups of networked repositories-rendered, -updated and -expanded compositions of relations of digital diagnosis-specific snippets, said individual groups rendering and successively updating and expanding said compilations of relations of digital diagnosis-specific snippets relating to said relational data on said financial transactions and related continuums in said relational databases in said AIR Pay, AIR Pays and chainlinks, adaptable security, and interactive live and real-time Ledgers and Graphs which optimizing people's health and standard of living at low costs, altering and lengthening lifespans, evolving human gene pools associating with said individual and individual groups, whereby said individual compilations of said relations of digital diagnosis-specific snippets rendering and successively updating and expanding the digital frameworks of AIR Pay and AIR Pays rendering and supporting said financial transactions in national currencies, digital currencies including fiat currency and cryptocurrencies for settling physical and digital debts, liabilities, credits and assets and for guaranteeing debts settlements and payments in said currencies and digital currencies in said transactions and e-commerce in said ledgers rendered by and among said networked repositories;
c. applying commercial statistical and actuarial software to render said instant and real-time statistical and actuarial analyses of said transactions of said financials, commodities, services and current and projected continuums related and linked to said individual groups of networked repositories-rendered, updated and expanded compilations of relations of digital diagnosis-specific snippets of relational chronologically ordered health and healthcare data and related relational profile data in the dynamic updating and expanding compilations of said relational data in the relational databases in the memories of, between and among said networked repositories;
d. rendering said individual groups of networked repositories-rendered, -updated and -expanded compilations of relations of digital diagnosis-specific snippets to render adaptable and versatile real-time security for protecting said transactions, AR Pay, Air Pays and chainlinks in, between and among said networked repositories from identities thefts, frauds, cyberattacks, hackings and corruptions;
e. rendering said compilations of relations of digital diagnosis-specific snippets for rendering peer-to-peer, device-to-device, devices-to-devices and device-to-servers transactions of AIR Pays in, between and among said clients and purveyors networked repositories;
f. rendering said individual groups of networked repositories-rendered, -updated and -expanded compilations of relations of digital diagnosis-specific snippets verifying individual changes, modifications and updates in said device identifications of individual networked devices in and among said individual groups of networked repositories in said relational databases in said chainlinks based on said processor units matching and verifying entire digital diagnosis-specific snippets in said individual subject networked repositories having said individual changes, modifications and updates within said compilations of relations of digital diagnosis-specific snippets in said individual groups of networked repositories-rendered AIR Pays whereby said innovative computer programs instructing said processor units to relate said individual subject networked repositories to said compilations of relations of digital diagnosis-specific snippets in said memories;
g. rendering said compilations of relations of snippets for rendering said networked repositories-specific custodies and guarantees of said national currencies and digital currencies in, between and among said individual groups of networked repositories;
h. storing and applying said thesaurus software networking said networked repositories for rendering and successively expanding and updating said relational databases, compilations, AIR Pay, AIR Pays and chainlinks, said networked repositories rendering said chainlinks by rendering, updating and expanding said compositions of relations of digital diagnosis-specific snippets comprising medical and surgical specialty-specific diagnoses and related and linked diagnosis-specific relational categories of chronologically ordered words, terms, terminologies and speeches in laypeople's languages and professionals' languages, images and videos and related said transactions and e-commerce of said commodities, services and continuums and related financial transactions;
j. attaching, updating and adjusting values, prices and costs in said national currencies and digital currencies of said commodities, services and continuums and said individual networked repositories- and individual groups of networked repositories-specific physical and digital debts, liabilities, credits and assets and guarantees and promissories for debts settlements and payments, said currencies and digital currencies including individual unique digital currencies keys, said processor units of said networked repositories processing said submissions and updates into said relational databases in memories in, between and among said networked devices;
j. successively assigning, rendering, updating said values, costs and prices in said currencies and digital currencies relating to said commodities, services and continuums;
k. rendering interactive dynamic real-time Comparative Values Prices Costs Ledgers and attached dynamic real-time Degree Distribution Graphs in the touchscreens and viewing screens of, between and among networked devices, said Comparative Values Prices Costs Ledgers rendering streaming, live and instant current and projected comparative data on said individual groups of networked repositories-specific financial transactions, accountings and tallies and associated transactions and e-commerce of said commodities, services and continuums in said AIR Pay, AIR Pays and said Degree Distribution Graphs rendering streaming, live and instant statistical and actuarial analyses of said relational data and the products, outcome and consequences of said Ledgers, said Ledgers and Graphs for optimizing people's health, healthcare, standard of living, values, costs, prices, lifespans and evolving human gene pools in, between and among said touchscreens and viewing screens of and among said networked devices, wherein said values encompass monetary, qualitative and intangible values of said commodities and services;
l. processing said Ledgers-rendered and Graphs-rendered relational data as feedbacks and adjustments on said intangible and tangible values, prices and costs related to digital snippets-specific useful and indicated commodities, services and continuums and related financial transactions including trades, exchanges, barters and auctions in said AIR Pay and AIR Pays in, between and among said networked repositories;
m. said interactive dynamic real-time Comparative Values Prices Costs Ledgers and in the attached dynamic real-time Degree Distribution Graphs for rendering, updating and expanding successive networked repositories-rendered transactions including trades, exchanges, barters and auctions wherein said interactive real-time Ledgers and Graphs for automatically rendering feedbacks on and adjusting said intangible and tangible values, prices and costs in said national currencies and digital currencies of said commodities, services and continuums and related financial transactions in said relational databases in said individual networked repositories- and individual groups of networked repositories-specific AIR Pays in AIR Pay;
n. applying said outcomes, products of said analytics as feedbacks and adjustments of said values, prices and costs of said commodities, services and useful and indicated continuums and related financial transactions in said compilations of relations of digital snippets-specific continuums in, between and among said individual networked repositories and individual groups of networked repositories;
o. applying said outcomes, products of said analytics as feedbacks and adjustments of said values, prices and costs of said commodities, services and useful and indicated continuums and related trades, exchanges, barters and auctions among said individual groups of clients and purveyors networked repositories;
p. calculating, accounting and tallying said financial transactions, accountings, transfers, payments, exchanges, barters, auctions, rendered feedbacks and analytics in, between and among said networked repositories-rendered AIR Pays in AIR Pay;
q. said compilations of relations of digital snippets, AIR lay, AIR Pays, interactive real-time Ledgers and Graphs and chainlinks optimizing people's health, healthcare, standard of living and the appropriate and lower values, costs and prices of related useful commodities, services and continuums, lifespans and human gene pools associating with said individual networked repositories and individual groups of networked repositories for global commerce and socioeconomic engineering based on said technologies;
r. rendering, streaming and displaying real-time and interactive AIR Pay, AIR Pays, digital snippets and products and outcomes of said processes in said touchscreens and viewing screens of said networked repositories.

10. The method according to claim 9 whereby said networked repositories and individual groups of networked repositories in and among said networked communication devices, said networked repositories comprising processor units and attached memories for storing said innovative computer programs hybridrizing said process units-rendered continuums of commodities and services with said genes-rendered compilations of relations of digital snippets by said innovative computer programs instructing said processor units rendering said hybridrizations to track, trend, effect and control said individual groups of networked repositories-rendered and -updated logistics rendering and controlling physical movements and locations and transactions of diverse industry-, industrial sector- and industrial sector categories-specific commodities and services and related financial transactions in and among said individual groups of networked repositories-specific AIR Pays, in individual groups of networked repositories-rendered, updated and expanded AIR Pay, Ledgers and Graphs, digital tokens and security and chainlinks.

11. The method according to claim 9 requires said networked repositories and individual groups of networked repositories to render, update and expand said relational databases in AIR Pays, AIR Pay, said digital tokens and security and chainlinks to comprise the following fundamental structures:
A representative relational profile data wherein biometrics data [*UB_data_p+n], [*UB_data_n+n] and [*UB_data_q+n] are almost always constant:
*unique key_p+n<>*unique key_n+n[created *TIMESTAMP_s]
*unique key_p+n<>*unique key_q+n[created *TIMESTAMP_n]
*unique key_q+n<>*unique key_q+n [created *TIMESTAMP_m]
Said networked repositories-related physicians' and purveyors' biometrics data and device and demographics data:
*unique key_p+1=[*physician_p+1_data]<[*UB_data_p+1]>[created *TIMESTAMP_a+1]
*unique key_p+1=[*physician_p+1_data]<[*UB_data_p+1]>[created *TIMESTAMP_a+2]
*unique key_p+2=[*physician_p+2_data]<[*UB_data_p+2]>[created *TIMESTAMP_a+4]
*unique key_p+n=[*physician_p+n_data]<[*UB_data_p+n]>[created *TIMESTAMP_a+n]
*unique key_p+1<[*UD_data_y+1]>[created *TIMESTAMP_a+1]
*unique key_p+1<[*UD_data_y+1]>[modified *TIMESTAMP_a+2]
*unique key_p++2<[*UD_data_y+2]>[modified *TIMESTAMP_a+4]
*unique key_p+n<[*UD_data_y+n]>[created *TIMESTAMP_a+n]
Said networked repositories-related patients' and clients' biometrics data and device and demographics data:
*unique key_n+1=[*patient_n+1_data]<[*UB_data_n+1]>[created *TIMESTAMP_a+6]
*unique key_n+1=[*patient_n+1_data]<[*UB_data_n+1]>[modified *TIMESTAMP_a+11]
*unique key_n+n=[*patient_n+n_data]<[*UB_data_n+n]>[created *TIMESTAMP_a+n]
*unique key_n+1<[*UD_data_y+10]>[created *TIMESTAMP_a+6]
*unique key_n+1<[*UD_data_y+133]>[modified *TIMESTAMP_a+11]
*unique key_n+n<[*UD_data_y+4536]>[created *TIMESTAMP_a+n]
Said networked repositories-related clients' and purveyors' biometrics data and device and demographics data:
*unique key_q+1=[*user_q+1_data]<[*UB_data_q+1]>[created *TIMESTAMP_a+1]
*unique key_q+1=[*user_q+1_data]<[*UB_data_q+1]>[modified *TIMESTAMP_a+3]
*unique key_q+2=[*user_q+2_data]<[*UB_data_q+2]>[created *TIMESTAMP_a+2]
*unique key_q+n=[*user_q+n_data]<[*UB_data_q+n]>[created *TIMESTAMP_a+n]
*unique key_q+1<[*UD_data_y+1]>[created *TIMESTAMP_a+1]
*unique key_q+1<[*UD_data_y+3]>[modified *TIMESTAMP_a+3]
*unique key_q+2<[*UD_data_y+2]>[created *TIMESTAMP_a+2]
*unique key_q+n<[*UD_data_y+5]>[created *TIMESTAMP_a+n]
Individual networked repositories-rendered, updated and expanded and individual groups of networked repositories-rendered, updated and expanded digital diagnosis-specific snippets of relational chronologically ordered health and healthcare data including values prices and costs and related relational profile data, accounting and tally functionalities are not shown here:
*unique key*_p+1<[*foreign key_m+1][*foreign key_m+2][*foreign key_m+3][created *TIMESTAMP_a+3]
*unique key*_p+1<[*foreign key_m+4][*foreign key_m+5][created *TIMESTAMP_a+111]
*unique key*_p+1<[*foreign key_m+n][created *TIMESTAMP_a+n]
*unique key*_n+1<[*foreign key_m+1][*foreign key_m+2][*foreign key_m+3][created *TIMESTAMP_a+14]
*unique key*_n+1<[*foreign key_m+4][*foreign key_m+5][created *TIMESTAMP_a+111]
*unique key*_n+1<[*foreign key_m+n][created *TIMESTAMP_a+n+11]
*unique key*_n+1<[*foreign key_o+1][*foreign key_o+2][*foreign key_o+3][created *TIMESTAMP_a°14]
*unique key*_n+1<[*foreign key_o+4][*foreign key_o+5][created *TIMESTAMP_a+111]
*unique key*_n+1<[*foreign key_o+n][created *TIMESTAMP_a+n+11]
*unique key_n+1<*diagnose_x+1_data<*RCOHH_data_x+1>[created *TIMESTAMP_a+2]
*unique key_m+2<*diagnose_x+2_data<*RCOHH_data_x+2>[created *TIMESTAMP_a+3]
*unique key_m+3<*diagnose_x+3_data<*RCOHH_data_x+3>[created *TIMESTAMP_a+4]
*unique key_m+4<*diagnose_x+4_data<*RCOHH_data_x+4>[created *TIMESTAMP_a+5]
*unique key_m+5<*diagnose_x+5_data<*RCOHH_data_x+5>[created *TIMESTAMP_a+6]
*unique key_m+n<*diagnose_x+n_data<*RCOHH_data_x+n>[created *TIMESTAMP_a+n]
[*foreign key_m+1]=*unique key_m+1<*diagnose_x+1_data<*RCOHH_data_x+1>[created *TIMESTAMP_a+2]
[*foreign key_m+2]=*unique key_m+2<diagnose_x+2_data<*RCOHH_data_x+2>[created *TIMESTAMP_a+3]
[*foreign key_m+3]=*unique key_m+3<*diagnose_x+3_data<*RCOHH_data_x+3>[created *TIMESTAMP_a+4]
[*foreign key_m+4]=*unique key_m+4<*diagnose_x+4_data<*RCOHH_data_x+4>[created *TIMESTAMP_a+5]
[*foreign key_m+5]=*unique key_m+5<*diagnose_x+5_data<*RCOHH_data_x+5>[created *TIMESTAMP_a+6]
[*foreign key_m+n]=*unique key_m+n<*diagnose_x+n_data<*RCOHH_data_x+n>[created *TIMESTAMP_a+n]
[*foreign key_o+1]*unique key_o+1<*STTPDEP_data_x+1<[*values_data_o+1]
[*prices_data_o+1][*costs_data_o+1]>[created *TIMESTAMP_a+2]
[*foreign key_o+2]=*unique key_o+2<*STTPDEP_data_x+2<[*values_data_o+2]
[*prices_data_o+2][*costs_data_o+2]>[created *TIMESTAMP_a+3]
[*foreign key_o+3]=*unique key_o+3<*STTPDEP_data_x+3<[*values_data_o+3]
[*prices_data_o+3][*costs_data_o+3]>[created *TIMESTAMP_a+4]
[*foreign key_o+4]*unique key_o+4<*STTPDEP_data_x+4<[*values_data_o+4]
[*prices_data_o+4][*costs_data_o+4]>[created *TIMESTAMP_a+5]
[*foreign key_o+5]=*unique key_o+5<*STTPDEP_data_x+5<[*values_data_o+5]
[*prices_data_o+5][*costs_data_o+5]>[created *TIMESTAMP_a+6]
[*foreign key_o+n]=*unique key_o+n<*STTPDEP_data_x+n<[*values_data_o+n]
[*prices_data_o+n][*costs_data_o+n]>[created *TIMESTAMP_a+n]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[values_data_o+1][*prices_data_o+1]
[*costs_data_o+1]>[created *TIMESTAMP_a+2]
*unique key_o+1>*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_p+1<[*foreign key_o+1]>[created *TIMESTAMP_a+2]
*unique key_p+1<[*foreign key_o+n]>[created *TIMESTAMP_a+5]
*unique key_p+n<[*foreign key_o+n]>[created TIMESTAMP_a+6]
*unique key_n+1<[*foreign key_o+1]>[created *TIMESTAMP_a+2]
*unique key_n+1<[*foreign key_o+n]>[created *TIMESTAMP_a+5]
*unique key_n+n<[*foreign key_o+n]>[created *TIMESTAMP_a+6]
Individual networked repositories-rendered, updated and expanded and individual networked repositories-rendered, updated and expanded commodities and services including values prices and costs:
*unique key_m+1<[*foreign key_o+1]>[created *TIMESTAMP_a+2]
*unique key_m+2<[*foreign key_o+2]>[created *TIMESTAMP_a+3]
*unique key_m+3<[*foreign key_o+3]>[created *TIMESTAMP_a+4]
*unique key_m+4<[*foreign key_o+4]>[created *TIMESTAMP_a+5]
*unique key_m+5<[*foreign key_o+5]>[created *TIMESTAMP_a+6]
*unique key_m+n<[*foreign key_o+n]>[created *TIMESTAMP_a+n]
*unique key*_q+1<[*foreign key_f+1]>[created *TIMESTAMP_c+2]
*unique key*_q+2<[*foreign key_f+2]>[created *TIMESTAMP_c+3]
*unique key*_q+1<[*foreign key_f+3]>[created *TIMESTAMP_c+4]
*unique key_m+4<[*foreign key_f+4]>[created *TIMESTAMP_c+5]
*unique key_m+5<[*foreign key_f+5]>[created *TIMESTAMP_c+6]
*unique key_m+n<[*foreign key_f+n]>[created *TIMESTAMP_c+n]
*unique key_q+1=[*user_n+1_data]<[*UB_data_x+1]<[*UD_data_y+1]>[created *TIMESTAMP_g+1]
*unique key_q+1=[*user_n+1_data]<[*UB_data_x+1]<[*UD_data_y+2]<[modified *TIMESTAMP_g+2]
*unique key_q+1=[*user_n+1_data]<[*UB_data_x+1]<[*UD_data_y+3]<[modified *TIMESTAMP_g+n]
*unique key_q+2=[*user_n+1_data]<[*UB_data_x+1]<[*UD_data_y+1]>[created *TIMESTAMP_g+4]
*unique key_q+3=[*user_n+1_data]<[*UB_data_x+1]<[*UD_data_y+1]>[created *TIMESTAMP_g+4]
*unique key_q+4=[*user_n+1_data]<[*UB_data_x+1]<[*UD_data_y+1]>[created *TIMESTAMP_g+8]
*unique key_q+n=[*user_n+1_data]<[*UB_data_x+1]<[*UD_data_y+1]>[created *TIMESTAMP_g+11]
*unique key_q+1<[*foreign key_e+1][created *TIMESTAMP_a+3]
*unique key_q+2<[*foreign key_e+4][created *TIMESTAMP_a+111]
*unique key_q+3<[*foreign key_e+n][created *TIMESTAMP_a+166]
*unique key_q+4<[*foreign key_e+n][created *TIMESTAMP_a+166]
*unique key_q+n<[*foreign key_e+n][created *TIMESTAMP_a+12]
[*foreign key_e+1]=*unique key_e+1<*commodities_services_data_x+1<[**values_data_x+1][**prices_data_x+1][**costs_data_x+1]>[created *TIMESTAMP_a+2]
[*foreign key_e+2]=*unique key_e+2<*commodities_services_data_x+2<[**values_data_x+2][**prices_data_x+2][*costs_data_x+2]>[created *TIMESTAMP_a+3]
[*foreign key_e+3]=*unique key_e+3<*commodities_services_data_x+3<[**values_data_x+3][**prices_data_x+3][**costs_data_x+3]>[created *TIMESTAMP_a+4]
[*foreign key_e+4]=*unique key_e+4<*commodities_services_data_x+4<[**values_data_x+4][**prices_data_x+4][**costs_data_x+4]>[created *TIMESTAMP_a+5]
[*foreign key_e+5]=*unique key_e+5<*commodities_services_data_x+5<[**values_data_x+5][**prices_data_x+5][**costs_data_x+5]>[created *TIMESTAMP_a+6]
[*foreign key_e+6]=*unique key_e+6<*commodities_services_data_x+6<[**values_data_x+6][**prices_data_x+6][**costs_data_x+6]>[created *TIMESTAMP_a+7]
[*foreign key_e+n]=*unique key_e+n<*commodities_services_data_x+n<[**values_data_x+n][**prices_data_x+n][**costs_data_x+n]>[created *TIMESTAMP_a+n]wherein said *commodities_services_data_x+n represent commodities and services in, but not limited to, health and healthcare commodities and services, devices and equipment, pharmaceuticals, food and beverages, financials, investment, estate planning and insurance, actuarial services, community and social services, acute, chronic, long-term and end-of-life care, legal services, communication, real estate, travel and transportation, hotel and hospitality industries; Individual groups of networked repositories-rendered, updated and expanded diverse digital snippets-specific continuums of commodities and services and continuums related to compilations of relations of digital diagnosis-specific snippets of relational chronologically ordered health and healthcare data and related relational profile data, said compilations of common relations of digital diagnosis-specific snippets for rendering said individual groups of networked repositories:
*unique key_n+1=[*patient_n+1_data]<[*UB_data_n+1]<[*UD_data_y+10]>[created *TIMESTAMP_a+6]
*unique key_n+1=[*patient_n+1_data]<[*UB_data_n+1]<[*UD_data_y+133]<[modified *TIMESTAMP_a+11]
*unique key_n+n=[*patient_n+n_data]<[*UB_data_n+n]<[*UD_data_y+4536]>[created *TIMESTAMP_a+n]
*unique key_n+1<[*foreign key_m+1]<[*foreign key_e+1][created *TIMESTAMP_a+2]
*unique key_n+1<[*foreign key_m+1]<[*foreign key_e+2][created *TIMESTAMP_a+4]
*unique key_n+1<[*foreign key_m+1]<[*foreign key_e+3][created *TIMESTAMP_a+6]
*unique key_n+1<[*foreign key_m+1]<[*foreign key_e+4][created *TIMESTAMP_a+8]
*unique key_n+1<[*foreign key_m+1]<[*foreign key_e+n][created *TIMESTAMP_a+10]
*unique key_n+1<[*foreign key_m+n]<[*foreign key_e+n][created *TIMESTAMP_a+n]
*unique key_n+n<[*foreign key_m+1]<[*foreign key_e+1][created *TIMESTAMP_a+3]
*unique key_n+n<[*foreign key_m+5]<[*foreign key_e+2][created *TIMESTAMP_a+5]
*unique key_n+n<[*foreign key_m+5]<[*foreign key_e+4][created *TIMESTAMP_a+7]
*unique key_n+n<[*foreign key_m+5]<[*foreign key_e+6][created *TIMESTAMP_a+9]
*unique key_n+n<[*foreign key_m+5]<[*foreign key_e+n][created *TIMESTAMP_a+n+1]
wherein a representative compilation of common relations of digital diagnosis-specific snippets of relational chronologically ordered health and healthcare data and related relational profile data are schematically represented by a common [*foreign key_m+1], said individual groups of networked repositories for rendering, updating and expanding AIR Pays, AIR Pay, said digital tokens and security and chainlinks based on common relations of digital diagnosis-specific snippets in said relations of compilations of relations:
*unique key_n+1<[*foreign key_m+1]<[*foreign key_e+1][created *TIMESTAMP_a+2]
*unique key_n+n<[*foreign key_m+1]<[*foreign key_e+1][created *TIMESTAMP_a+3]

12. In the system of networked communication devices for rendering logistics for rendering, tracking, trending, effecting and controlling said networked repositories-rendered transactions and physical movements and locations of diverse industry-, industrial sector- and industrial sector categories-specific commodities and services and related financial transactions, said networked devices comprise individual networked repositories and individual groups of clients and purveyors networked repositories comprising said processor units for rendering a plurality of interactive electronic industry-, industrial sector- and categories-specific platforms for rendering, updating and expanding compilations of relations of digital diagnosis-specific snippets of relational chronologically ordered medical, surgical, users' health and healthcare data and related clients' and purveyors' biometrics and demographics data and device identifications and said devices' identification and related industry-, industrial sector- and industrial sector categories-specific commodities, services and said digital snippets-specific continuums of said commodities and services and related financial transactions including accountings, transfers, payments, exchanges, barters and auctions and tallies related tangible and intangible values, prices and costs in national currencies and digital currencies including cryptocurrencies in the adaptable and expanding relational databases henceforth known as AIR Pay in the memories of said networked repositories wherein said individual groups for rendering interactive dynamic real-time Comparative Values Prices Costs Ledgers to which attached said dynamic real-time Degree Distribution Graphs in the touchscreens and viewing screens of, between and among worked devices, said Comparative Values Prices Costs Ledgers for rendering streaming, live and instant current and projected comparative data on said individual groups of networked repositories-specific financial transactions and associated transactions and e-commerce of said commodities, services and continuums in said AIR Pay and AIR Pays and said Degree Distribution Graphs for rendering streaming, live and instant statistical and actuarial analyses of said relational data in said AIR Pays and said individual groups of individual networked repositories-rendered products, outcomes and consequences of said comparative data and related analytics of said Ledgers and Graphs as feedbacks and adjustments of said values, prices and costs in said national currencies and digital currencies and said commodities, services and continuums and related financial transactions in said relational databases in said individual networked repositories- and individual groups of networked repositories-specific AIR Pays in said AIR Pay, and said individual networked repositories- and individual groups of networked repositories-rendered updated and expanded compilations of relations of digital diagnosis-specific snippets for rendering, updating and expanding said AIR Pay and AIR Pays in, between and among said networked repositories, wherein said networked repositories for rendering, updating and expanding AIR Pay, individual networked repositories-specific AIR Pays and individual groups of clients and purveryors networked repositories-specific AIR Pays based on said processor units-compiled compilations of common relations of digital diagnosis-specific snippets in and among said networked repositories, wherein said compilations of relations of digital diagnosis-specific snippets for rendering, updating and expanding said transactions AIR Pay, AIR Pays, chainlinks, for rendering adaptable and versatile real-time security for protecting said transactions, AIR Pay, AIR Pays, chainlinks and for rendering and guaranteeing the custodians of said currencies and digital currencies in, between and among said networked repositories, the method of networked repositories-rendered Abiotic Intelligence-rendered logistics tracking, trending, effecting and controlling said transactions and physical movements and locations of diverse industry-, industrial sector- and industrial sector categories-specific commodities and services and related financial transactions, said method comprising diverse digital snippets-specific continuums of said commodities and services and related financial transactions including exchanges, trades, barters and auctions, in national currencies and digital currencies including cryptocurrencies, related to relations of compilations of relations and compilations of relations of digital diagnosis-specific snippets of relational chronologically ordered medical and surgical health and healthcare data and related relational profile data in said relational databases in said networked repositories-rendered Abiotic Intelligence-Rendered digital ledgers accounting and tallying said transactions in said memories in, between and among clients and purveyors networked repositories henceforth known as AIR Pay, said individual groups of networked repositories rendering, updating and expanding interactive dynamic real-time Comparative Values Prices Costs Ledgers to which attached said dynamic real-time Degree Distribution Graphs in the touchscreens and viewing screens of, between and among networked devices; said Comparative Values Prices Costs Ledgers rendering, updating and expanding successive interactive and dynamic streaming real-time current and projected successive comparative data on said individual groups of networked repositories-specific financial transactions and associated transactions and e-commerce of said continuums in said AIR Pays; and successive diverse interactive and dynamic streaming real-time Degree Distribution Graphs rendering streaming and real-time statistical and actuarial analyses of said relational data as feedbacks and adjustments on said intangible and tangible values, prices and costs related to digital snippets-specific useful and indicated commodities, services and continuums and related financial transactions including trades, exchanges, barters and auctions in said AIR Pay and AIR Pays in, between and among said networked repositories, in said networked repositories, the innovative computer programs including the thesaurus software instructing said processor units rendering and successively updating and expanding said compilations of relations of digital snippets forming the electronic frameworks in said relational databases in said AIR Pay and, based on said compilations of common elations of digital snippets, said individual groups of networked repositories rendering and successively updating and expanding AIR Pays, said compilations of relations of digital snippets rendering and guaranteeing said individual networked repositories-specific and individual groups of networked repositories-specific custodians and guarantees of said national currencies and digital currencies including said cryptocurrencies, said compilations of relations of digital diagnosis-specific snippets rendering peer-to-peer, device-to-device, devices-to-devices and device-to-servers transactions of AIR Pays in said AIR Pay in, between and among said clients and purveyors networked repositories, said individual groups of networked repositories-rendered, -updated and -expanded compilations of relations of digital diagnosis-specific snippets for verifying individual changes, modifications and updates in said device identifications of individual networked devices in and among said individual groups of networked repositories in said relational databases in said chainlinks based on said processor units matching and verifying entire digital diagnosis-specific snippets in said individual subject networked repositories having said individual changes, modifications and updates within said compilations of relations of digital diagnosis-specific snippets in said individual groups of networked repositories-rendered AIR Pays whereby said innovative computer programs instructing said processor units to relate said individual subject networked repositories to said compilations of relations of digital diagnosis-specific snippets in said memories, the method comprises processes of:
networking said networked devices comprising networked servers, mobile communication devices and desktops, said networked repositories comprising individual client networked repositories and individual purveyors networked repositories, said networked repositories rendering, successively updating and expanding said individual groups of clients and purveyors networked repositories-rendered, updated and expanded AIR Pay and AIR Pays whereby said individual groups of clients and purveyors networked repositories rendering, updating and expanding said compilations of common relations of snippets and related said financial transactions, transactions of commodities, services and continuums, said networked repositories comprising said processor units and memories for rendering and storing said innovative computer programs, said relational databases including said compilations and successive compilations in said AIR Pay and related products and outcomes of said Ledgers and Graphs and and chainlinks, said networked devices comprising said touchscreens and viewing screens rendering and displaying said a plurality of interactive electronic industry-, industrial sector- and categories-specific platforms, said compilations of relations of digital snippets, said AIR Pay, and individual groups of networked repositories-specific AIR Pays, interactive real-time Ledgers and Graphs and edited and adaptable individual groups of networked repositories-specific AIR Pays; said networked repositories having and using a plurality of commercial software rendering said users' biometrics data—facial including selfies biometrics, retina biometrics, voice biometrics, fingerprints, genetic fingerprints, amino acid sequences in DNA sequences, laboratory results of body fluids and tissues, sequences of binary numbers coding laboratory images such as, digital x-ray, MRI, CTScan and ultrasound;
said networked repositories having and using a plurality of near field technologies rendering and successively expanding and updating said relational databases, chainlinks and security in, between and among said networked repositories, a plurality of software Apps for rendering said system in and among said networked repositories;
said networked devices implementing a plurality of commercial software for rendering said users' biometrics data such as, but not limited to, facial including selfies biometrics, retina biometrics, voice biometrics, fingerprints, genetic fingerprints, amino acid sequences in DNA sequences, laboratory results of body fluids and tissues, sequences of binary numbers coding laboratory images such as, digital x-ray, MRI, CTScan and ultrasound, a plurality of near field technologies for rendering and successively expanding and updating said relational databases, chainlinks and security in, between and among said networked repositories, and a plurality of software Apps for rendering said system in and among said networked repositories;
said networked repositories-rendered Abiotic Intelligence tracking, trending, effecting and controlling relational databases to comprise compilations of relational financial transactions relating to said transactions and commerce of goods and services in diverse industries and industrial sectors henceforth commodities and services and said diverse continuums of commodities and services related to said networked repositories-rendered, updated and expanded compilations of relations of digital diagnosis-specific snippets of relational chronologically ordered health and healthcare data and related relational profile data in said relational databases whereby said individual groups of said networked repositories for rendering, updating and expanding said continuums to comprise relations of digital snippets-specific commodities, services and continuums being processed by said processor units and stored in said memories of said networked repositories henceforth collectively known as networked repositories-rendered Abiotic Intelligence-Rendered digital ledgers accounting and tallying said transactions of financials, commodities, services and continuums in, between and among clients and purveyors networked repositories henceforth known as AIR Pay;
whereby said networked repositories rendering, updating and expanding individual networked repositories-specific AIR Pays and individual groups of said individual networked repositories-specific AIR Pays comprising said compilations of con mon relations of digital diagnosis-specific snippets from said AIR Pay being rendered and viewed in and among the touchscreens and viewing screens of networked devices, and inherent in said AIR Pay and AIR Pays are said individual digital snippets and individual compilations of relations of said digital snippets rendering and safeguarding the custodians of said currencies and digital currencies in, between and among said networked repositories whereby said digital snippets rendering said transactions and rendering adaptable and versatile real-time security protecting said transactions, AIR Pay, AIR Pays, chainlinks and system in, between and among said networked repositories;
whereby said individual groups of individual networked repositories rendering, updating and expanding interactive dynamic real-time Comparative Values Prices Costs Ledgers to which attached said dynamic real-time Degree Distribution Graphs in the touchscreens and viewing screens of, between and among networked devices rendering, updating and expanding successive interactive and dynamic streaming real-time Comparative Values Prices Costs Ledgers rendering streaming, live and instant current and projected successive comparative data on said individual groups of networked repositories-specific financial transactions and associated transactions and e-commerce of said continuums in said AIR Pay, AIR Pays and successive diverse interactive and dynamic streaming real-time Degree Distribution Graphs rendering streaming, live and instant statistical and actuarial analyses of said data;
whereby said individual groups of networked repositories feedback and adjust said values, prices and costs in said national currencies and digital currencies of said commodities, services and continuums and related financial transactions in said relational databases in said AIR Pay and AIR Pays based on said products, outcomes and consequences of said analyses relating to said individual groups of networked repositories-rendered compilations of relations of snippets in said relational databases in said AIR Pay and AIR Pays by said processor units whereby said individual networked repositories- and individual groups of networked repositories-specific AIR Pays to optimize people's health, healthcare, standard of living, values, costs, prices, lifespans and evolving human gene pools in, between and among said touchscreens and viewing screens of and among said networked devices, wherein said values encompass monetary, qualitative and intangible values of said commodities and services, said method comprises processes of:
said individual networked repositories and individual groups of individual networked repositories including clients and purveyors networked repositories rendering, updating and expanding said AIR Pay and AIR Pays wherein said networked repositories for rendering a plurality of interactive electronic industry-, industrial sector- and categories-specific platforms for rendering, updating, and expanding said compilations of relations of digital diagnosis-specific snippets of relational chronologically ordered medical, surgical, users' health and healthcare data and related relational profile data and related industry-, industrial sector- and categories-specific said commodities, services and said digital snippets-specific continuums of said commodities and services and related financial transactions, and wherein said individual groups and clients and purveyors for rendering, a plurality of interactive electronic industry-, industrial sector- and categories-specific platforms for rendering, updating and expanding said compilations of common relations of digital diagnosis-specific snippets of relational chronologically ordered medical, surgical, users' health and healthcare data and related users' biometrics and demographics data and device identifications and related industry-, industrial sector- and categories-specific said commodities, services and continuums and related financial transactions in, between and among said networked repositories wherein said continuums comprising said digital snippets-specific commodities and services;
said compilations of relations of snippets rendering peer-to-peer, device-to-device, devices-to-devices and device-to-servers transactions in, between and among clients and purveyors networked repositories, said individual groups of networked repositories-rendered, -updated and -expanded said compilations of relations of snippets rendering adaptable and versatile real-time security protecting said transactions, AIR Pay, AIR Pays, chainlinks and system in, between, and among said networked repositories from identities thefts, frauds, cyberattacks, hackings and corruptions, said compilations of relations of snippets are inherent in said relational databases in said chainlinks whereby said compilations of relations of snippets rendering and guaranteeing said networked repositories-specific custodians and guarantees of said national currencies and digital currencies;
said innovative computer programs instructing said processor units and attached memories of said networked repositories and individual groups of networked repositories to render, update and expand said compilations of relations of digital snippets in said AIR Pay, AIR Pays in said AIR PAY, and how to calculate, perform, account and tally said financial transactions, accountings, transfers, payments, exchanges, barters and auctions in, between and among said networked repositories and said analytics by said Ledgers and Graphs and feedbacks in said AIR Pay and AIR Pays, said processor units and attached memories; and
said innovative computer programs instructing said processor units attached to said memories of said networked repositories and individual groups of networked repositories whereby said processor units:
a1. rendering an electronic platform for rendering and updating said biometrics and demographics data physicians, patients, clients, purveyors, companies, corporations and third parties and networked device identifications, storing said relational data in said memories;
a2. rendering said relational profile data in said continuums of diverse industry-, industrial sector- and categories-specific commodities and services as said relational profile data in, between and among said touchscreens and viewing screens of said networked devices and individual groups of networked devices;
b. rendering said individual networked repositories-rendered, -updated and -expanded and individual groups of networked repositories-rendered, -updated and -expanded compositions of relations of digital diagnosis-specific snippets, said individual groups rendering and successively updating and expanding said compilations of relations of digital diagnosis-specific snippets relating to said relational data on said financial transactions and related continuums in said relational databases in said AIR Pay, AIR Pays and chainlinks, adaptable security, and interactive live and real-time Ledgers and Graphs which optimizing people's health and standard of living at low costs, altering and lengthening lifespans, evolving human gene pools associating with said individual and individual groups, whereby said individual compilations of said relations of digital diagnosis-specific snippets rendering and successively updating and expanding the digital frameworks of AIR Pay and AIR Pays rendering and supporting said financial transactions in national currencies, digital currencies including fiat currency and cryptocurrencies for settling physical and digital debts, liabilities, credits and assets and for guaranteeing debts settlements and payments in said currencies and digital currencies in said transactions and e-commerce in said ledgers rendered by and among said networked repositories;
c. applying commercial statistical and actuarial software to render said instant and real-time statistical and actuarial analyses of said transactions of said financials, commodities, services and current and projected continuums related and linked to said individual groups of networked repositories-rendered, updated and expanded compilations of relations of digital diagnosis-specific snippets of relational chronologically ordered health and healthcare data and related users' biometrics and demographics data and device identifications in the dynamic updating and expanding compilations of said relational data in the relational databases in the memories of, between and among said networked repositories;
d. rendering said individual groups of networked repositories-rendered, -updated and -expanded compilations of relations of digital diagnosis-specific snippets to render adaptable and versatile real-time security for protecting said transactions, AIR Pay, Air Pays and chainlinks in, between and among said networked repositories from identities thefts, frauds, cyberattacks, hackings and corruptions;
e. rendering said compilations of relations of digital diagnosis-specific snippets for rendering peer-to-peer, device-to-device, devices-to-devices and device-to-servers transactions of AIR Pays in, between and among said clients and purveyors networked repositories;
f. rendering said individual groups of networked repositories-rendered, -updated and -expanded compilations of relations of digital diagnosis-specific snippets verifying individual changes, modifications and updates in said device identifications of individual networked devices in and among said individual groups of networked repositories in said relational databases in said chainlinks based on said processor units matching and verifying entire digital diagnosis-specific snippets in said individual subject networked repositories having said individual changes, modifications and updates within said compilations of relations of digital diagnosis-specific snippets in said individual groups of networked repositories-rendered AIR Pays whereby said innovative computer programs instructing said processor units to relate said individual subject networked repositories to said compilations of relations of digital diagnosis-specific snippets in said memories;
g. rendering said compilations of relations of snippets for rendering said networked repositories-specific custodies and guarantees of said national currencies and digital currencies in, between and among said individual groups of networked repositories;
h. storing and applying said thesaurus software networking said networked repositories for rendering and successively expanding and updating said relational databases, compilations, AIR Pay, AIR Pays and chainlinks, said networked repositories rendering said chainlinks by rendering, updating and expanding said compositions of relations of digital diagnosis-specific snippets comprising medical and surgical specialty-specific diagnoses and related and linked diagnosis-specific relational categories of chronologically ordered words, terms, terminologies and speeches in laypeople's languages and professionals' languages, images and videos and related said transactions and e-commerce of said commodities, services and continuums and related financial transactions;
i. attaching, updating and adjusting values, prices and costs in said national currencies and digital currencies of said commodities, services and continuums and said individual networked repositories- and individual groups of networked repositories-specific physical and digital debts, liabilities, credits and assets and guarantees and promissories for debts settlements and payments, said currencies and digital currencies including individual unique digital currencies keys, said processor units of said networked repositories processing said submissions and updates into said relational databases in memories in, between and among said networked devices;
j. successively assigning, rendering, updating said values, costs and prices in said currencies and digital currencies relating to said commodities, services and continuums;
k. rendering interactive dynamic real-time Comparative Values Prices Costs Ledgers and attached dynamic real-time Degree Distribution Graphs in the touchscreens and viewing screens of, between and among networked devices, said Comparative Values Pries Costs Ledgers rendering streaming, live and instant current and projected comparative data on said individual groups of networked repositories-specific financial transactions, accountings and tallies and associated transactions and e-commerce of said commodities, services and continuums in said AIR Pay, AIR Pays and said Degree Distribution Graphs rendering streaming, live and instant statistical and actuarial analyses of said relational data and the products, outcomes and consequences of said Ledgers, said Ledgers and Graphs for optimizing people's health, healthcare, standard of living, values, costs, prices, lifespans and evolving human gene pools in, between and among said touchscreens and viewing screens of and among said networked devices, wherein said values encompass monetary, qualitative and intangible values of said commodities and services;
l. processing said Ledgers-rendered and Graphs-rendered relational data as feedbacks and adjustments on said intangible and tangible values, prices and costs related to digital snippets-specific useful and indicated commodities, services and continuums and related financial transactions including trades, exchanges, barters and auctions in said AIR Pay and AIR Pays in, between and among said networked repositories;
m. said interactive dynamic real-time Comparative Values Prices Costs Ledgers and in the attached dynamic Degree Distribution Graphs for rendering, updating and expanding successive networked repositories-rendered transactions including trades, exchanges, barters and auctions wherein said interactive real-time Ledgers and Graphs for automatically rendering feedbacks on and adjusting said intangible and tangible values, prices and costs in said national currencies and digital currencies of said commodities, services and continuums and related financial transactions in said relational databases in said individual networked repositories- and individual groups of networked repositories-specific AIR Pays in AIR Pay;
n. applying said outcomes, products of said analytics as feedbacks and adjustments of said values, prices and costs of said commodities, services and useful and indicated continuums and related financial transactions in said compilations of relations of digital snippets-specific continuums in, between and among said individual networked repositories and individual groups of networked repositories;
o. applying said outcomes, products of said analytics as feedbacks and adjustments of said values, prices and costs of said commodities, services and useful and indicated continuums and related trades, exchanges, barters and auctions among said individual groups of clients and purveyors networked repositories;
p. calculating, accounting and tallying said financial transactions, accountings, transfers payments, exchanges, barters, auctions, rendered feedbacks and analytics in, between and among said networked repositories-rendered AIR Pays in AIR Pay;
q. said compilations of relations of digital snippets, AIR Pay, AIR Pays in interactive real-time Ledgers and Graphs and chainlinks optimizing people's health, healthcare, standard of living and the appropriate and lower vales, costs and prices if related useful commodities, services and continuums, lifespans and human gene pools associating with said individual networked repositories and individual groups of networked repositories for global commerce and socioeconomic engineering based on said technologies;
r. rendering, streaming and displaying real-time and interactive AIR Pay, AIR Pays, digital snippets and products and outcomes of said processes in said touchscreens and viewing screens of said networked repositories.

13. The method according to claim 12 wherein said innovative computer programs attaching to and instructing said processor units to:
a1. render first interactive electronic platforms rendering and updating relational profile data comprising biometrics and demographics and professions data and networked device identifications of physicians, patients, clients, purveyors, companies, corporations and third parties, storing said relational profile data in the memories of and among said networked repositories;
a2. apply and update said relational profile data in said continuums of diverse industry-, industrial sector- and categories-specific commodities and services related to said relational profile data, render said relational profile data in, between and among said touchscreens and viewing screens of said networked devices and individual groups of networked devices having said compilations of common relations of digital diagnosis-specific snippets;
a3. render second interactive electronic platforms rendering and updating financial relational data comprising individual networked repositories- and individual groups of networked repositories-specific physical and digital debts, liabilities, credits and assets and guarantees and promissories for debts settlements and payments, said currencies and digital currencies including individual unique digital currencies keys relating to said relational profile data whereby said compilations of relations rendering and updating said networked repositories-specific custodians and guarantors of said national currencies and digital currencies relating to said relational profile data, said processor units processing said relational data as submissions and updates into said relational databases in said memories;
c1. render third interactive electronic platforms attaching, updating and adjusting individual values prices and costs, in said national currencies and digital currencies, assigned to said diverse industry-, industrial sector- and categories-specific commodities and services relating to said relational profile data, storing said relations in said relational databases in said memories;
c2. render fourth interactive electronic platforms rendering and displaying said relational values, prices and costs and diverse industry-, industrial sector- and categories-specific commodities and services in individual interactive electronic templates for rendering and updating said relational values, prices and costs and commodities and services relating to said relational profile data, storing said relations in said relational databases in said memories;
d1. render fifth interactive electronic platforms rendering and displaying medical and surgical specialties-specific diagnoses and diagnoses-specific relational categories of diverse clinical and hospital office Visit and consultation services, chief Complaints, symptoms, signs, laboratory tests, pharmaceuticals, diagnoses, related and linked diagnoses, differential diagnoses, treatments, procedures, devices, equipments, recommendations, referrals, hospitals, clinical notes and messages, drug allergy, food allergy, wherein said diagnoses-specific chief Complaints, symptoms, signs and treatments are expressed in chronologically ordered words, terms, terminologies, images, videos and speeches based on the actual chronologically expressions of diseases and disorders; applying said innovative thesaurus software to implement and display said words, terms, terminologies and speeches in laypeople's languages and professionals' languages applicable to said physicians, patients, clients and purveyors, the successive outcomes and products successively rendered by said interactive electronic platforms comprise said individual networked repositories-rendered, updated and expanded compilations of relations of digital diagnosis-specific snippets of relational chronologically ordered health and healthcare data to said relational profile data, storing said relations in said relational databases in said memories;
d2. whereby, render said individual groups of networked repositories-rendered, updated and expanded compilations of relations of digital diagnosis-specific snippets of relational chronologically ordered health and healthcare data relating to said relational profile data by said rendering networked repositories searching common relations of said digital diagnosis-specific snippets in and among said digital diagnosis-specific snippets in said relational databases whereby said processor units rendering and successively updating and expanding said individual groups of networked repositories including said rendering networked repositories having said common relations of said digital diagnosis-specific snippets;
e1. render sixth interactive electronic platforms rendering said individual groups of physicians and patients networked repositories rendering updating and expanding compilations of relations of digital diagnosis-specific snippets of relational chronologically ordered health and healthcare data including commodities and services relating to said relational profile data, storing said relations in said relational databases in said memories;
e2. render sixth interactive electronic platforms rendering said individual groups of physicians and patients networked repositories rendering updating and expanding compilations of relations of related and linked digital diagnosis-specific snippets of relational chronologically ordered health and healthcare data including commodities and services relating to said relational profile data, storing said relations in said relational databases in said memories;
e3. calculate, account and tally said financial transactions, accountings, transfers, payments and credits relating to said relational values, prices and costs and health and healthcare commodities and services relating to said relational profile data in and among said compilations of relations of digital diagnosis-specific snippets in said relational databases, storing said compilations in said relational databases in said memories;
f1. render said sixth interactive electronic platforms rendering said individual clients and purveyors networked repositories rendering and relating continuums of diverse industry-. industrial sector- and categories-specific commodities and services, relating to said relational profile data, which are specifically related by said processor units to said relational chronologically ordered health and healthcare data in said digital diagnosis-specific snippets, consequently, said successively rendered compilations of outcomes are stored in said memories;
f2. calculate, account and tally said financial transactions, accountings, transfers, payments and credits relating to said relational values, prices and costs of said continuums related to said successively rendered compilations of outcomes, storing said accountings and tallies in said relational databases in said memories;
g1. render said seventh interactive electronic platform rendering said interactive dynamic real-time Comparative Values Prices Costs Ledgers and attached dynamic real-time Degree Distribution Graphs rendering, updating and expanding said successive networked repositories-rendered transactions including trades, exchanges, barters and auctions wherein said interactive real-time Ledgers comparing values, prices and costs and cost-effectiveness of said commodities, services and current and projected continuums in and among said individual groups of networked repositories, and, applying statistics and actuarial data, said Degree Distribution Graphs execute instant and real-time statistical and actuarial analyses of said transactions and analyses of financial transactions, usefulness, indications and cost-effectiveness commodities, services and current and projected continuums in, between and among said individual groups of networked repositories;
g2. apply said outcomes, products of said analytics as feedbacks and adjustments of said values, prices and costs of said commodities, services and useful and indicated commodities, services in said continuums and related financial transactions in said compilations of relations of digital snippets-specific continuums in, between and among said individual networked repositories and individual groups of networked repositories;
g3. apply said outcomes, products of said analytics as feedbacks and adjustments of said values, prices and costs of said commodities, services and useful and indicated continuums and related trades, exchanges, barters and auctions among said individual groups of clients and purveyors networked repositories;
h. render, stream and display interactive real-time AIR Pays, relations of digital snippets, continuums and said products and outcomes, and feedback and adjustments in interactive real-time electronic multi-dimensional visual-hierarchied ledgers and graphs said touchscreens and viewing screens of said networked repositories; and
i. render a plurality of interactive digital templates for rendering said platforms-rendered, updated and expanded relational databases in the touchscreens and viewing screens of said networked devices.

14. The method according to claim 13 wherein said individual groups of networked repositories-rendered Abiotic Intelligence-rendered logistics tracking, trending, effecting and controlling physical movements and locations and said transactions of diverse industries-, industrial sectors- and categories-specific commodities and services and related financial transactions, said processor units instructed by said innovative computer programs to render, update and expand said relations of compilations of relations and compilations of relations in said relational databases comprising AIR Pays, AIR Pay, said digital tokens and security and chainlinks whose fundamental structures are as follow:
A representative relational profile data wherein biometrics data [*UB_data_p+n], [*UB_data_n+n] and [*UB_data_q+n] are almost always constant:
*unique key_p+n<>*unique key_n+n[created *TIMESTAMP_s]
*unique key_p+n<>*unique key_q+n[created *TIMESTAMP_n]
*unique key_q+n<>*unique key_q+n[created *TIMESTAMP_m]
Said networked repositories-related physicians' and purveyors' biometrics data and device and demographics data:
*unique key_p+1=[*physician_p+1_data]<[*UB_data_p+1]>[created *TIMESTAMP_a+1]
*unique key_p+1=[*physician_p+1_data]<[*UB_data_p+1]>[created *TIMESTAMP_a+2]
*unique key_p+2=[*physician_p+2_data]<[*UB_data_p+2]>[created *TIMESTAMP_a+4]
*unique key_p+n=[*physician_p+n_data]<[*UB_data_p+n]>[created *TIMESTAMP_a+n]
*unique key_p+1<[*UD_data_y+1]>[created *TIMESTAMP_a+1]
*unique key_p+1<[*UD_data_y+1]>[modified *TIMESTAMP_a+2]
*unique key_p+2<[*UD_data_y+2]>[modified *TIMESTAMP_a+4]
*unique key_p+n<[*UD_data_y+n]>[created *TIMESTAMP_a+n]
Said networked repositories-related patients' and clients' biometrics data and device and demographics data:
*unique key_n+1=[*patient_n+1_data]<[*UB_data_n+1]>[created *TIMESTAMP_a+6]
*unique key_n+1=[*patient_n+1_data]<[*UB_data_n+1]>[modified *TIMESTAMP_a+11]
*unique key_n+n=[*patient_n+n_data]<[*UB_data_n+n]>[created *TIMESTAMP_a+n]
*unique key_n+1<[*UD_data_y+10]>[created *TIMESTAMP_a+6]
*unique key_n+1<[*UD_data_y+133]>[modified *TIMESTAMP_a+11]
*unique key_n+n<[*UD_data_y+4536]>[created *TIMESTAMP_a+n]
Said networked repositories-related clients' and purveyors' biometrics data and device and demographics data:
*unique key_q+1=[*user_q+1_data]<[*UB_data_q+1]>[created *TIMESTAMP_a+1]
*unique key_q+1=[*user_q+1_data]<[*UB_data_q+1]>[modified *TIMESTAMP_a+3]
*unique key_q+2=[*user_q+2_data]<[*UB_data_q+2]>[created *TIMESTAMP_a+2]
*unique key_q+n=[*user_q+n_data]<[*UB_data_q+n_]>[created *TIMESTAMP_a+n]
*unique key_q+1<[*UD_data_y+1]>[created *TIMESTAMP_a+1]
*unique key_q+1<[*UD_data_y+3]>[modified *TIMESTAMP_a+3]
*unique key_q+2<[*UD_data_y+2]>[created *TIMESTAMP_a+2]
*unique key_q+n<[*UD_data_y+5]>[created *TIMESTAMP_a+n]
Individual networked repositories-rendered, updated and expanded and individual groups of networked repositories-rendered, updated and expanded digital diagnosis-specific snippets of relational chronologically ordered health and healthcare data including values prices and costs and related relational profile data, accounting and tally functionalities are not shown here:
*unique key*_p+1<[foreign key_m+1][*foreign key_m+2][*foreign key_m+3][created *TIMESTAMP_a+3]
*unique key*_p+1<[*foreign key_m+4][*foreign key_m+5][created *TIMESTAMP_a+111]
*unique key*_p+1<[*foreign key_m+n][created *TIMESTAMP_a+n]
*unique key*_n+1<[*foreign key_m+1][*foreign key_m+2][*foreign key_m+3][created *TIMESTAMP_a+14]
*unique key*_n+1<[*foreign key_m+4][*foreign key_m+5][created *TIMESTAMP_a+111]
*unique key*_n+1<[*foreign key_m+n][created *TIMESTAMP_a+n+11]
*unique key*_n+1<[*foreign key_o+1][*foreign key_o+2][*foreign key_o+3][created *TIMESTAMP_a+14]
*unique key*_n+1<[*foreign key_o+4][*foreign key_o+5][created *TIMESTAMP_a+111]
*unique key*_n+1<[*foreign key_o+n][created *TIMESTAMP_a+n+11]
*unique key_m+1<*diagnose_x+1_data<*RCOHH_data_x+1>[created *TIMESTAMP_a2]
*unique key_m+2<*diagnose_x+2_data<*RCOHH_data_x+2>[created *TIMESTAMP_+3]
*unique key_m+3<*diagnose_x+3_data<*RCOHH_data_x+3>[created *TIMESTAMP_a+4]
*unique key_m+4<*diagnose_x+4_data<*RCOHH_data_x+4>[created *TIMESTAMP_a+5]
*unique key_m+5<*diagnose_x+5_data<*RCOHH_data_x+5>[created *TIMESTAMP_a+6]
*unique key_m+n<*diagnose_x+n_data<*RCOHH_data_x+n>[created *TIMESTAMP_a+n]
[*foreign key_m+1]=*unique key_m+1<*diagnose_x+1_data<*RCOHH_data_x+1>[created *TIMESTAMP_a+2]
[*foreign key_m+2]=*unique key_m+2<*diagnose_x+2_data<*RCOHH_data_x+2>[created *TIMESTAMP_a+3]
[*foreign key_m+3]=*unique key_m+3<*diagnose_x+3_data<*RCOHH_data_x+3>[created *TIMESTAMP_a+4]
[*foreign key_m+4]=*unique key_m+4<*diagnose_x+4_data<*RCOHH_data_x+4>[created *TIMESTAMP_a+5]
[*foreign key_m+5]=*unique key_m+5<*diagnose_x+5_data<*RCOHH_data_x+5>[created *TIMESTAMP_a+6]
[*foreign key_m+n]=*unique key_m+n<*diagnose_x+n_data<*RCOHH_data_x+n>[created *TIMESTAMP_a+n]
[*foreign key_o+1]=*unique key_o+1<*STTPDEP_data_x+1<[*values_data_o+1]
[*prices_data_o+1][*costs_data_o+1]>[created *TIMESTAMP_a+2]
[*foreign key_o+2]=*unique key_o+2<*STTPDEP_data_x+2<[*values_data_o+2]
[*prices_data_o+2][*costs_data_o+2]>[created *TIMESTAMP_a+3]
[*foreign key_o+3]=*unique key_o+3<*STTPDEP_data_x+3<[*values_data_o+3]
[*prices_data_o+3][*costs_data_o+3]>[created *TIMESTAMP_a+4]
[*foreign key_o+4]=*unique key_o+4<*STTPDEP_data_x+4<[*values_data_o+4]
[*prices_data_o+4][*costs_data_o+4]>[created *TIMESTAMP_a+5]
[*foreign key_o+5]=*unique key_o+5<*STTPDEP_data_x+5<[*values_data_o+5]
[*prices_data_o+5][*costs_data_o+5]>[created *TIMESTAMP_a+6]
[*foreign key_o+n]=*unique key_o+n<*STTPDEP_data_x+n<[*values_data_o+n]
[*prices_data_o+n][costs_data_o+n]>[created *TIMESTAMP_a+n]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_o+1<*STTPDEP_data_x+1<[*values_data_o+1][*prices_data_o+1]
[*costs_data_o+1]>[created *TIMESTAMP_a+2]
*unique key_p+1<[*foreign key_o+1]>[created *TIMESTAMP_a+2]
*unique key_p+1<[*foreign key_o+n]>[created *TIMESTAMP_a+5]
*unique key_p+n<[*foreign key_o+n]>[created *TIMESTAMP_a+6]
*unique key_n+1<[*foreign key_o+1]>[created *TIMESTAMP_a+2]
*unique key_n+1<[*foreign key_o+n]>[created *TIMESTAMP_a+5]
*unique key_n+n<[*foreign key_o+n]>[created *TIMESTAMP_a+6]
Individual networked repositories-rendered, updated and expanded and individual networked repositories-rendered, updated and expanded commodities and services including values prices and costs:
*unique key_m+1<[*foreign key_o+1]>[created *TIMESTAMP_a+2]
*unique key_m+2<[*foreign key_o+2]>[created *TIMESTAMP_a+3]
*unique key_m+3<[*foreign key_o+3]>[created *TIMESTAMP_a+4]
*unique key_m+4<[*foreign key_o+4]>[created *TIMESTAMP_a+5]
*unique key_m+5<[*foreign key_o+5]>[created *TIMESTAMP_a+6]
*unique key_m+n<[*foreign key_o+n]>[created *TIMESTAMP_a+n]
*unique key*_q+1<[*foreign key_f+1]>[created *TIMESTAMP_c+2]
*unique key*_q+2<[*foreign key_f+2]>[created *TIMESTAMP_c+3]
*unique key*_q+1<[*foreign key_f+3]>[created *TIMESTAMP_c+4]
*unique key_m+4<[*foreign key_f+4]>[created *TIMESTAMP_c+5]
*unique key_m+5<[*foreign key_f+5]>[created *TIMESTAMP_c+6]
*unique key_m+n<[*foreign key_f+n]>[created *TIMESTAMP_c+n]
*unique key_q+1=[*user_n+1_data]<[*UB_data_x+1]<[*UD_data_y+1]>[created *TIMESTAMP_g+1]
*unique key_q+1=[*user_n+1_data]<[*UB_data_x+1]<[*UD_data_y+2]<[modified *TIMESTAMP_g+2]
*unique key_q+1=[*user_n+1_data]<[*UB_data_x+1]<[*UD_data_y+3]<[modified *TIMESTAMP_g+n]
*unique key_q+2=[*user_n+1_data]<[*UB_data_x+1]<[*UD_data_y+1]>[created *TIMESTAMP_g+4]
*unique key_q+3=[*user_n+1_data]<[*UB_data_x+1]<[*UD_data_y+1]>[created *TIMESTAMP_g+4]
*unique key_q+4<[*user_n+1_data]<[*UB_data_x+1]<[*UD_data_y+1]>[created *TIMESTAMP_g+8]
*unique key_q+n=[*user_n+1_data]<[*UB_data_x+1]<[*UD_data_y+1]>[created *TIMESTAMP_g+11]
*unique key_q+1<[*foreign key_e+1][created *TIMESTAMP_a+3]
*unique key_q+2<[*foreign key_e+4][created *TIMESTAMP_a+111]
*unique key_q+3<[*foreign key_e+n][created *TIMESTAMP_a+166]
*unique key_q+4<[*foreign key_e+n][created *TIMESTAMP_a+166]
*unique key_q+n<[*foreign key_e+n][created *TIMESTAMP_a+12]
[*foreign key_e+1]=*unique key_e+1<*commodities_services_data_x+1<[**values_data_x+1][**prices_data_x+1][**costs_data_x+1]>[created *TIMESTAMP_a+2]
[*foreign key_e+2]=*unique key_e+2<*commodities_services_data_x+2<[**values_data_x+2][**prices_data_x+2][**costs_data_x+2]>[created *TIMESTAMP_a+3]
[*foreign key_e+3]=*unique key_e+3<*commodities_services_data_x+3<[**values_data_x+3][**prices_data_x+3][**costs_data_x+3]>[created *TIMESTAMP_a+4]
[*foreign key_e+4]=*unique key_e+4<*commodities_services_data_x+4<[**values_data_x+4][**prices_data_x+4][**costs_data_x+4]>[created *TIMESTAMP_a+5]
[*foreign key_e+5]=*unique key_e+5<*commodities_services_data_x+5<[**values_data_x+5][**prices_data_x+5][**costs_data_x+5]>[created *TIMESTAMP_a+6]
[*foreign key_e+6]=*unique key_e+6<*commodities_services_data_x+6<[**values_data_x+6][**prices_data_x+6][**costs_data_x+6]>[created *TIMESTAMP_a+7]
[*foreign key_e+n]=*unique key_e+n<*commodities_services_data_x+n<[**values_data_x+n][**prices_data_x+n][**costs_data_x+n]>[created *TIMESTAMP_a+n]
wherein said *commodities_services_data_x+n represent commodities and services in, but not limited to, health and healthcare commodities and services, devices and equipment, pharmaceuticals, food and beverages, financials, investment, estate planning and insurance, actuarial services, community and social services, acute, chronic, long-term and end-of-life care, legal services, communication, real estate, travel and transportation, hotel and hospitality industries;
Individual groups of networked repositories-rendered, updated and expanded diverse digital snippets-specific continuums of commodities and services and continuums related to compilations of relations of digital diagnosis-specific snippets of relational chronologically ordered health and healthcare data and related relational profile data, said compilations of common relations of digital diagnosis-specific snippets for rendering said individual groups of networked repositories:
*unique key_n+1=[*patient_n+1_data]<[*UB_data_n+1]<[*UD_data_y+10]>[created *TIMESTAMP_a+6]
*unique key_n+1=[*patient_n+1_data]<[*UB_data_n+1]<[*UD_data_y+133]<[modified *TIMESTAMP_a+11]
*unique key_n+n=[*patient_n+n_data]<[*UB_data_n+n]<[*UD_data_y+4536]>[created *TIMESTAMP_a+n]
*unique key_n+1<[*foreign key_m+1]<[*foreign key_e+1][created *TIMESTAMP_a+2]
*unique key_n+1<[*foreign key_m+1]<[*foreign key_e+2][created *TIMESTAMP_a+4]
*unique key_n+1<[*foreign key_m+1]<[*foreign key_e+3][created *TIMESTAMP_a+6]
*unique key_n+1<[*foreign key_m+1]<[*foreign key_e+4][created *TIMESTAMP_a+8]
*unique key_n+1<[*foreign key_m+1]<[*foreign key_e+n][created *TIMESTAMP_a+10]
*unique key_n+1<[*foreign key_m+n]<[*foreign key_e+n][created *TIMESTAMP_a+n]
*unique key_n+n<[*foreign key_m+1]<[*foreign key_e+1][created *TIMESTAMP_a+3]
*unique key_n+n<[*foreign key_m+5]<[*foreign key_e+2][created *TIMESTAMP_a+5]
*unique key_p+n<[*foreign key_m+5]<[*foreign key_e+4][created *TIMESTAMP_a+7]
*unique key_n+n<[*foreign key_m+5]<[*foreign key_e+6][created *TIMESTAMP_a+9]
*unique key_n+n<[*foreign key_m+5]<[*foreign key_e+n][created *TIMESTAMP_a+n+1]
where said *user represent people and entities associating with said networked devices such as clients, purveryors, physicians, patients, payees, payers companies, corporations, third parties, etc. Said a, c, e, f, g, n, m, o, p, q, r, s, x, y, z each of which represents 0 people, 0 uses or 0 commodities and services and 1, 2, 3, 4 . . . infinite number of people, users or commodities and services; all people become patients and clients and some people are physicians and purveyors;
“*” and “**” represent “a, each, every”; *UB_data represent a, each, every or all unique biometrics data of people and users, *UD_data represent a, each, every or all device data and demographics data of people and users; *RCOHH_data represents diagnosis*-specific chronologically ordered health and healthcare relational data; *STTPDEP represents a plurality of office Visit and consultation services, laboratory tests, treatments, procedures, devices, equipments, pharmaceuticals and others some of which are associated with said *values *costs and *prices; and same concept applied to said commodities, services and continuums associated with said **values, **costs, and **prices; categories, subcategories, headings or headers, topics or types in medicine, surgeries and health-care as described in the preferred embodiments specialty*- and diagnosis*-specific Drug Allergy, Food Allergy, Chief Complaints, Symptoms, signs, laboratory tests, diagnoses, differential diagnoses, treatments, procedures, pharmaceuticals, diverse levels of office Visit and consultations, recommendations, referrals, hospitals, Clinics, notes, messages; said [*values] [*prices][*costs][*values][**prices][**costs] of said commodities and services and continuums in said financial transactions, accountings, transfers, payments and credits are transacted in national currencies, digital currencies including fiat currency and cryptocurrencies; said symbols are arbritary and may not conform to the standard computer programming languages.
